(19) United States
(12) Patent Application Publication
(10) Pub. No.: US 2016/0083805 A1
(43) Pub. Date: Mar. 24, 2016

(54) METHODS AND COMPOSITIONS FOR ASSESSMENT OF PULMONARY FUNCTION AND DISORDERS

(71) Applicant: SYNERGENZ BIOSCIENCE LTD, Hong Kong (CN)

(72) Inventor: Robert Peter Young, Auckland (NZ)

(21) Appl. No.: 14/958,582

(22) Filed: Dec. 3, 2015

Related U.S. Application Data
(63) Continuation of application No. 13/677,221, filed on Nov. 14, 2012, now abandoned.
Application No. 13/677,221 is a continuation of application No. 11/874,187, filed on Oct. 17, 2007, now abandoned.

(30) Foreign Application Priority Data
Oct. 17, 2006 (MZ) 550643
Nov. 22, 2006 (NZ) 551534
Dec. 7, 2006 (NZ) 551883
Apr. 23, 2007 (NZ) 554707
Jul. 31, 2007 (NZ) 560262
Jul. 31, 2007 (NZ) 560263

(51) Int. Cl. C12Q 1/68

(52) U.S. Cl. CPC C12Q 1/6886; C12Q 2600/158; C12Q 2600/156

(57) ABSTRACT

The present invention provides methods for the assessment of risk of developing lung cancer in smokers and non-smokers using analysis of genetic polymorphisms. The present invention also relates to the use of genetic polymorphisms in assessing a subject's risk of developing lung cancer, and the suitability of a subject for an intervention in respect of lung cancer. Nucleotide probes and primers, kits, and microarrays suitable for such assessment are also provided.

### CROSS-REFERENCES

The present application is a continuation of U.S. patent application Ser. No. 13/677,221 filed on Nov. 14, 2012, which is a continuation of U.S. patent application Ser. No. 11/874,187 filed Oct. 17, 2007, which claims priority to New Zealand Patent Application No. 550643 filed on Oct. 17, 2006, New Zealand Patent Application No. 551534 filed on Nov. 22, 2006, New Zealand Patent Application No. 551883 filed on Dec. 7, 2006, New Zealand Patent Application No. 554707 filed on Apr. 23, 2007, New Zealand Patent Application No. 560262 filed on Jul. 31, 2007, and New Zealand Patent Application No. 560263, filed on Jul. 31, 2007, each of the foregoing is incorporated herein by reference in its entirety.

### FIELD OF THE INVENTION

The present invention is concerned with methods for assessment of pulmonary function and/or disorders, and in particular for assessing risk of developing lung cancer in smokers and non-smokers using analysis of genetic polymorphisms.

### BACKGROUND OF THE INVENTION

Lung cancer is the second most common cancer and has been attributed primarily to cigarette smoking. Other factors contributing to the development of lung cancer include occupational exposure, genetic factors, radon exposure, exposure to other aero-pollutants and possibly dietary factors (Alberg A J, et al., 2003). Non-smokers are estimated to have a one in 400 risk of lung cancer (0.25%). Smoking increases this risk by approximately 40 fold, such that smokers have a one in 10 risk of lung cancer (10%) and in long-term smokers the life-time risk of lung cancer has been reported to be as high 10-15% (Schwartz A G. 2004). Genetic factors are thought to play some part as evidenced by a weak familial tendency (among smokers) and the fact that only the minority of smokers get lung cancer. It is generally accepted that the majority of this genetic tendency comes from low penetrant high frequency polymorphisms, that is, polymorphisms which are common in the general population that in context of chronic smoking exposure contribute collectively to cancer development (Schwartz A G. 2004, Wu X et al., 2004). Several epidemiological studies have reported that impaired lung function (Anthonisen N R. 1989, Skillrud D M. 1986, Tockman M S et al., 1987, Kuller L H, et al., 1990, Nomura A, et al., 1991) or symptoms of obstructive lung disease (Mayne S T, et al., 1999) are independent risk factors for lung cancer and are possibly more relevant than smoking exposure dose.

Despite advances in the treatment of airways disease, current therapies do not significantly alter the natural history of lung cancer, which may include metastasis and progressive loss of lung function causing respiratory failure and death. Although cessation of smoking may be expected to reduce this decline in lung function, it is probable that if this is not achieved at an early stage, the loss is considerable and symptoms of worsening breathlessness likely cannot be averted. Analogous to the discovery of serum cholesterol and its link to coronary artery disease, there is a need to better understand the factors that contribute to lung cancer so that tests that identify at risk subjects can be developed and that new treatments can be discovered to reduce the adverse effects of lung cancer. The early diagnosis of lung cancer or of a propensity to developing lung cancer enables a broader range of prophylactic or therapeutic treatments to be employed than can be employed in the treatment of late stage lung cancer. Such prophylactic or early therapeutic treatment is also more likely to be successful, achieve remission, improve quality of life, and/or increase lifespan.

To date, a number of biomarkers useful in the diagnosis and assessment of propensity towards developing various pulmonary disorders have been identified. These include, for example, single nucleotide polymorphisms including the following: A-82G in the promoter of the gene encoding human macrophage elastase (MMP12); T→C within codon 10 of the gene encoding transforming growth factor beta (TGFβ); C+760G of the gene encoding superoxide dismutase 3 (SOD3); T-1296C within the promoter of the gene encoding tissue inhibitor of metalloproteinase 3 (TIMP3); and polymorphisms in linkage disequilibrium with these polymorphisms, as disclosed in PCT International Application PCT/NZ02/00106 (published as WO 02/099134 and incorporated herein in its entirety).

It would be desirable and advantageous to have additional biomarkers which could be used to assess a subject's risk of developing pulmonary disorders such as lung cancer, or a risk of developing lung cancer-related impaired lung function, particularly if the subject is a smoker.

It is primarily to such biomarkers and their use in methods to assess risk of developing such disorders that the present invention is directed.

### SUMMARY OF THE INVENTION

The present invention is primarily based on the finding that certain polymorphisms are found more often in subjects with lung cancer than in control subjects. Analysis of these polymorphisms reveals an association between polymorphisms and the subject's risk of developing lung cancer.

Thus, according to one aspect there is provided a method of determining a subject's risk of developing lung cancer comprising analysing a sample from said subject for the presence or absence of one or more polymorphisms selected from the group consisting of:
Ser307Ser G/T (rs1056503) in the X-ray repair complementing defective repair in Chinese hamster cells 4 gene (XRCC4),
A/T c74delA in the gene encoding cytochrome P450 polypeptide CYP3A43 (CYP3A43),
A/C (rs2279115) in the gene encoding B-cell CLL/lymphoma 2 (BCL2),
A/G at +3100 in the 3′UTR (rs2317676) of the gene encoding Integrin beta 3 (ITGB3),
−3714 G/T (rs6413429) in the gene encoding Dopamine transporter 1 (DAT1),
A/G (rs1139417) in the gene encoding Tumor necrosis factor receptor 1 (TNFR1),
C/Del (rs1799732) in the gene encoding Dopamine receptor D2 (DRD2),
C/T (rs763110) in the gene encoding Fas ligand (FasL), or
C/T (rs5743836) in the gene encoding Toll-like receptor 9 (TLR9),

wherein the presence or absence of said polymorphism is indicative of the subject's risk of developing lung cancer.

This polymorphism can be detected directly or by detection of one or more polymorphisms which are in linkage disequilibrium with one or more of said polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

The lung cancer may be non-small cell lung cancer including adenocarcinoma and squamous cell carcinoma, or small cell lung cancer, or may be a carcinoid tumor, a lymphoma, or a metastatic cancer.

The method can additionally comprise analysing a sample from said subject for the presence or absence of one or more further polymorphisms selected from the group consisting of:
R19W A/G (rs10115703) in the gene encoding Cerberus 1 (Cer 1);
K3326X A/T (rs11571833) in the breast cancer 2 early onset gene (BRCA2);
V433M A/G (rs2306022) in the gene encoding Integrin alpha-11;
E375G T/C (rs7214723) in the gene encoding Calcium/calmodulin-dependent protein kinase kinase 1 (CAMKK1); or
−81 C/T (rs 2273953) in the 5′ UTR of the gene encoding Tumor protein P73 (P73).

Again, detection of the one or more further polymorphisms may be carried out directly or by detection of polymorphisms in linkage disequilibrium with the one or more further polymorphisms.

The presence of one or more polymorphisms selected from the group consisting of:
the E375G T/C TT genotype in the gene encoding CAMKK1;
the −81 C/T (rs 2273953) CC genotype the gene encoding P73;
the A/C (rs2279115) AA genotype in the gene encoding BCL2;
the +3100 A/G (rs2317676) AG or GG genotype in the gene encoding ITGB3;
the C/Del (rs1799732) CDel or DelDel genotype in the gene encoding DRD2; or
the C/T (rs763110) TT genotype in the gene encoding FasL,
   may be indicative of a reduced risk of developing lung cancer.

The presence of one or more polymorphisms selected from the group consisting of:
the R19W A/G AA or GG genotype in the gene encoding Cer 1;
the Ser307Ser G/T GG or GT genotype in the XRCC4 gene;
the K3326X A/T AT or TT genotype in the BRCA2 gene;
the V433M A/G AA genotype in the gene encoding Integrin alpha-11;
the A/T c74delA AT or TT genotype in the gene encoding CYP3A43;
the −3714 G/T (rs6413429) GT or TT genotype in the gene encoding DAT1;
the A/G (rs1139417) AA genotype in the gene encoding TNFR1; or
the C/T (rs5743836) CC genotype in the gene encoding TLR9,
   may be indicative of an increased risk of developing lung cancer.

The methods of the invention are particularly useful in smokers (both current and former).

It will be appreciated that the methods of the invention identify two categories of polymorphisms—namely those associated with a reduced risk of developing lung cancer (which can be termed “protective polymorphisms”) and those associated with an increased risk of developing lung cancer (which can be termed “susceptibility polymorphisms”).

Therefore, the present invention further provides a method of assessing a subject's risk of developing lung cancer, said method comprising:

determining the presence or absence of at least one protective polymorphism associated with a reduced risk of developing lung cancer; and

in the absence of at least one protective polymorphism, determining the presence or absence of at least one susceptibility polymorphism associated with an increased risk of developing lung cancer;

wherein the presence of one or more of said protective polymorphisms is indicative of a reduced risk of developing lung cancer, and the absence of at least one protective polymorphism in combination with the presence of at least one susceptibility polymorphism is indicative of an increased risk of developing lung cancer.

Preferably, the at least one protective polymorphism selected from the group consisting of:
the E375G T/C TT genotype in the gene encoding CAMKK1;
the −81 C/T (rs 2273953) CC genotype the gene encoding P73;
the A/C (rs2279115) AA genotype in the gene encoding BCL2;
the +3100 A/G (rs2317676) AG or GG genotype in the gene encoding ITGB3;
the C/Del (rs1799732) CDel or DelDel genotype in the gene encoding DRD2; or
the C/T (rs763110) TT genotype in the gene encoding Fas ligand.

The at least one susceptibility polymorphism may be selected from the group consisting of:
the R19W A/G AA or GG genotype in the gene encoding Cer 1;

the Ser307Ser G/T GG or GT genotype in the XRCC4 gene;
the K3326X A/T AT or TT genotype in the BRCA2 gene;
the V433M A/G AA genotype in the gene encoding Integrin alpha-11;
the A/T c74delA AT or TT genotype in the gene encoding CYP3A43;
the −3714 G/T (rs6413429) GT or TT genotype in the gene encoding DAT1;
the A/G (rs1139417) AA genotype in the gene encoding TNFR1; or
the C/T (rs5743836) CC genotype in the gene encoding TLR9.

In a preferred form of the invention the presence of two or more protective polymorphisms is indicative of a reduced risk of developing lung cancer.

In a further preferred form of the invention the presence of two or more susceptibility polymorphisms is indicative of an increased risk of developing lung cancer.

In still a further preferred form of the invention the presence of two or more protective polymorphims irrespective of the presence of one or more susceptibility polymorphisms is indicative of reduced risk of developing lung cancer.

In another aspect, the invention provides a method of determining a subject's risk of developing lung cancer, said method comprising obtaining the result of one or more genetic tests of a sample from said subject, and analysing the result for the presence or absence of of one or more polymorphisms selected from the group consisting of:
Ser307Ser G/T in the X-ray repair complementing defective repair in Chinese hamster cells 4 gene;
A/T c74delA in the gene encoding cytochrome P450 polypeptide CYP3A43,
A/C (rs2279115) in the gene encoding B-cell CLL/lymphoma 2,
A/G at +3100 in the 3′UTR (rs2317676) of the gene encoding Integrin beta 3,
−3714 G/T (rs6413429) in the gene encoding Dopamine transporter 1,
A/G (rs1139417) in the gene encoding Tumor necrosis factor receptor 1,
C/Del (rs1799732) in the gene encoding Dopamine receptor D2,
C/T (rs763110) in the gene encoding Fas ligand,
C/T (rs5743836) in the gene encoding Toll-like receptor 9,

or one or more polymorphisms in linkage disequilibrium with this polymorphism;

wherein a result indicating the presence or absence of one or more of said polymorphisms is indicative of the subject's risk of developing lung cancer.

The method can additionally comprise obtaining the result of one or more genetic tests of a sample from said subject, and analysing the result for the presence or absence of one or more further polymorphisms selected from the group consisting of:
R19W A/G in the gene encoding Cerberus 1;
K3326X A/T in the breast cancer 2 early onset gene;
V433M A/G in the gene encoding Integrin alpha-11;
E375G T/C in the gene encoding Calcium/calmodulin-dependent protein kinase kinase 1; or
−81 C/T (rs 2273953) in the 5′ UTR of the gene encoding Tumor protein P73.

Again, the presence or absence may be determined directly or by determining the presence or absence of polymorphisms in linkage disequilibrium with the one or more further polymorphisms.

In a further aspect there is provided a method of determining a subject's risk of developing lung cancer comprising the analysis of two or more polymorphisms selected from the group consisting of:
R19W A/G in the gene encoding Cerberus 1;
Ser307Ser G/T in the X-ray repair complementing defective repair in Chinese hamster cells 4 gene;
K3326X A/T in the breast cancer 2 early onset gene;

V433M A/G in the gene encoding Integrin alpha-11; or
E375G T/C in the gene encoding Calcium/calmodulin-dependent protein kinase kinase 1;
A/T c74delA in the gene encoding cytochrome P450 polypeptide CYP3A43,
A/C (rs2279115) in the gene encoding B-cell CLL/lymphoma 2,
A/G at +3100 in the 3′UTR (rs2317676) of the gene encoding Integrin beta 3,
−3714 G/T (rs6413429) in the gene encoding Dopamine transporter 1,
A/G (rs1139417) in the gene encoding Tumor necrosis factor receptor 1,
C/Del (rs1799732) in the gene encoding Dopamine receptor D2,
C/T (rs763110) in the gene encoding Fas ligand,
C/T (rs5743836) in the gene encoding Toll-like receptor 9,
−81 C/T (rs 2273953) in the 5′ UTR of the gene encoding Tumor protein P73, or

one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
V433M A/G (rs2306022) in the gene encoding ITGA11;

or one or more polymorphisms in linkage disequilibrium with any one or more of these polymorphisms.

In one embodiment of the methods and uses of the present invention each of the following polymorphisms are selected:
Rsa 1 C/T (rs2031920) in the gene encoding CYP 2E1;
−133 G/C (rs360721) in the promoter of the gene encoding Interleukin-18;
−251 A/T (rs4073) in the gene encoding Interleukin-8;
−511 A/G (rs 16944) in the gene encoding Interleukin 1B;
V433M A/G (rs2306022) in the gene encoding ITGA11;
Arg 197 Gln A/G (rs 1799930) in the gene encoding N-acetylcysteine transferase 2;
Ala 15 Thr A/G (rs4934) in the gene encoding α1-antichymotrypsin;
R19W A/G (rs 10115703) in the gene encoding Cerberus 1;
−3714 G/T (rs6413429) in the gene encoding DAT1;
A/G (rs1139417) in the gene encoding TNFR1;
C/T (rs5743836) in the gene encoding TLR9;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
−751 G/T (rs 13181) in the promoter of the gene encoding XPD;
Phe 257 Ser C/T (rs3087386) in the gene encoding REV1;
C/T (rs763110) in the gene encoding FasL;

or one or more polymorphisms in linkage disequilibrium with any one or more of these polymorphisms.

In various embodiments, any one or more of the above methods comprises the step of analysing the amino acid present at a position mapping to codon 19 of the gene encoding Cer 1.

The presence of tryptophan at said position is indicative of an increased risk of developing lung cancer.

The presence of arginine at said position is indicative of reduced risk of developing lung cancer.

In various embodiments, any one or more of the above methods comprises the step of analysing the amino acid present at a position mapping to codon 3326 in the BRCA2 gene.

The presence of lysine at said position is indicative of reduced risk of developing lung cancer.

The presence of a truncated gene product of 3325 amino acids is indicative of an increased risk of developing lung cancer.

In various embodiments, any one or more of the above methods comprises the step of analysing the amino acid present at a position mapping to codon 433 in the gene encoding Integrin alpha-11.

The presence of methionine at said position is indicative of an increased risk of developing lung cancer.

The presence of valine at said position is indicative of reduced risk of developing lung cancer.

In various embodiments, any one or more of the above methods comprises the step of analysing the amino acid present at a position mapping to codon 375 in the gene encoding CAMKK1.

The presence of glycine at said position is indicative of an increased risk of developing lung cancer.

The presence of glutamate at said position is indicative of reduced risk of developing lung cancer.

In a preferred form of the invention the methods as described herein are performed in conjunction with an analysis of one or more risk factors, including one or more epidemiological risk factors, associated with a risk of developing lung cancer. Such epidemiological risk factors include but are not limited to smoking or exposure to tobacco smoke, age, sex, and familial history of lung cancer.

In a further aspect, the invention provides for the use of at least one polymorphism in the assessment of a subject's risk of developing lung cancer, wherein the at least one polymorphism is selected from the group consisting of;
Ser307Ser G/T in the X-ray repair complementing defective repair in Chinese hamster cells 4 gene;
A/T c74delA in the gene encoding cytochrome P450 polypeptide CYP3A43,
A/C (rs2279115) in the gene encoding B-cell CLL/lymphoma 2,
A/G at +3100 in the 3′UTR (rs2317676) of the gene encoding Integrin beta 3,
−3714 G/T (rs6413429) in the gene encoding Dopamine transporter 1,
A/G (rs1139417) in the gene encoding Tumor necrosis factor receptor 1,
C/Del (rs1799732) in the gene encoding Dopamine receptor D2,
C/T (rs763110) in the gene encoding Fas ligand, or
C/T (rs5743836) in the gene encoding Toll-like receptor 9,

or one or more polymorphisms in linkage disequilibrium with said polymorphism.

Optionally, said use may be in conjunction with the use of at least one further polymorphism selected from the group consisting of:
R19W A/G in the gene encoding Cerberus 1 (Cer 1);
K3326X A/T in the breast cancer 2 early onset gene (BRCA2);
V433M A/G in the gene encoding Integrin alpha-11;
E375G T/C in the gene encoding Calcium/calmodulin-dependent protein kinase kinase 1 (CAMKK1);
−81 C/T (rs 2273953) in the 5′ UTR of the gene encoding Tumor protein P73;

or one or more polymorphisms which are in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
V433M A/G (rs2306022) in the gene encoding ITGA11;

or one or more polymorphisms in linkage disequilibrium with any one or more of these polymorphisms.

In one embodiment of the methods and uses of the present invention each of the following polymorphisms are selected:
Rsa 1 C/T (rs2031920) in the gene encoding CYP 2E1;
−133 G/C (rs360721) in the promoter of the gene encoding Interleukin-18;
−251 A/T (rs4073) in the gene encoding Interleukin-8;
−511 A/G (rs 16944) in the gene encoding Interleukin 1B;
V433M A/G (rs2306022) in the gene encoding ITGA11;
Arg 197 Gln A/G (rs 1799930) in the gene encoding N-acetylcysteine transferase 2;
Ala 15 Thr A/G (rs4934) in the gene encoding α1-antichymotrypsin;
R19W A/G (rs 10115703) in the gene encoding Cerberus 1;
−3714 G/T (rs6413429) in the gene encoding DAT1;
A/G (rs1139417) in the gene encoding TNFR1;
C/T (rs5743836) in the gene encoding TLR9;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;

−751 G/T (rs 13181) in the promoter of the gene encoding XPD;
Phe 257 Ser C/T (rs3087386) in the gene encoding REV1;
C/T (rs763110) in the gene encoding FasL;

or one or more polymorphisms in linkage disequilibrium with any one or more of these polymorphisms.

In another aspect the invention provides a set of nucleotide probes and/or primers for use in the preferred methods of the invention herein described. Preferably, the nucleotide probes and/or primers are those which span, or are able to be used to span, the polymorphic regions of the genes. Also provided are one or more nucleotide probes and/or primers comprising the sequence of any one of the probes and/or primers herein described, including any one comprising the sequence of any one of SEQ.ID.NO. 1 to 72, more preferably any one of SEQ.ID.NO. 1 to 10 or any one of SEQ.ID.NO. 26 to 43.

In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention, which microarray comprises a substrate presenting antibodies capable of binding to a product of expression of a gene the expression of which is upregulated or downregulated when associated with a susceptibility or protective polymorphism as described herein.

In a further aspect the present invention provides a method treating a subject having an increased risk of developing lung cancer comprising the step of replicating, genotypically or phenotypically, the presence and/or functional effect of a protective polymorphism in said subject.

In yet a further aspect, the present invention provides a method of treating a subject having an increased risk of developing lung cancer, said subject having a detectable susceptibility polymorphism which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In yet a further aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a susceptibility or protective polymorphism which has been determined to be associated with the upregulation or downregulation of expression of a gene; and

measuring the expression of said gene following contact with said candidate compound,

wherein a change in the level of expression after the contacting step as compared to before the contacting step is indicative of the ability of the compound to modulate the expression and/or activity of said gene.

Preferably, said cell is a human lung cell which has been pre-screened to confirm the presence of said polymorphism.

Preferably, said cell comprises a susceptibility polymorphism associated with upregulation of expression of said gene and said screening is for candidate compounds which downregulate expression of said gene.

Alternatively, said cell comprises a susceptibility polymorphism associated with downregulation of expression of said gene and said screening is for candidate compounds which upregulate expression of said gene.

In another embodiment, said cell comprises a protective polymorphism associated with upregulation of expression of said gene and said screening is for candidate compounds which further upregulate expression of said gene.

Alternatively, said cell comprises a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a gene, the expression of which is upregulated or downregulated when associated with a susceptibility or protective polymorphism but which in said cell the expression of which is neither upregulated nor downregulated; and

measuring the expression of said gene following contact with said candidate compound,

wherein a change in the level of expression after the contacting step as compared to before the contacting step is indicative of the ability of the compound to modulate the expression and/or activity of said gene.

Preferably, expression of the gene is downregulated when associated with a susceptibility polymorphism once said screening is for candidate compounds which in said cell, upregulate expression of said gene.

Preferably, said cell is a human lung cell which has been pre-screened to confirm the presence, and baseline level of expression, of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from lung cancer to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism which when present either upregulates or downregulates expression of said gene such that the physiological active concentration of the expressed gene product is outside said normal range, wherein the detection of the presence of said polymorphism is indicative of the subject likely responding to said treatment.

In still a further aspect, the present invention provides a method of assessing a subject's suitability for an intervention that is diagnostic of or therapeutic for a disease, the method comprising:

a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;
and

b) providing a distribution of net scores for disease sufferers and non-sufferers wherein the net scores for disease sufferers and non-sufferers are or have been determined in the same manner as the net score determined for said subject;

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

In one embodiment, the intervention is a diagnostic test for said disease.

In another embodiment, the intervention is a therapy for said disease, more preferably a preventative therapy for said disease.

Preferably, the disease is lung cancer, more preferably the disease is lung cancer and the protective and susceptibility polymorphisms are selected from the group consisting of:
the −133 G/C polymorphism in the Interleukin-18 gene;
the −1053 C/T polymorphism in the CYP 2E1 gene;
the Arg197Gln polymorphism in the NAT2 gene;
the −511 G/A polymorphism in the Interleukin 1B gene;
the Ala 9 Thr polymorphism in the Anti-chymotrypsin gene;
the S allele polymorphism in the Alpha1-antitrypsin gene;
the −251 A/T polymorphism in the Interleukin-8 gene;
the Lys 751 gln polymorphism in the XPD gene;
the +760 G/C polymorphism in the SOD3 gene;
the Phe257Ser polymorphism in the REV gene;
the Z alelle polymorphism in the Alpha1-antitrypsin gene;
the R19W A/G polymorphism in the Cerberus 1 (Cer 1) gene;
the Ser307Ser G/T polymorphism in the XRCC4 gene;
the K3326X A/T polymorphism in the BRCA2 gene;
the V433M A/G polymorphism in the Integrin alpha-11 gene;
the E375G T/C polymorphism in the CAMKK1 gene;
the A/T c74delA polymorphism in the gene encoding cytochrome P450 polypeptide CYP3A43,
the A/C (rs2279115) polymorphism in the gene encoding B-cell CLL/lymphoma 2,
the A/G at +3100 in the 3′UTR (rs2317676) polymorphism of the gene encoding Integrin beta 3,
the −3714 G/T (rs6413429) polymorphism in the gene encoding Dopamine transporter 1,
the A/G (rs1139417) polymorphism in the gene encoding Tumor necrosis factor receptor 1,
the C/Del (rs1799732) polymorphism in the gene encoding Dopamine receptor D2,
the C/T (rs763110) polymorphism in the gene encoding Fas ligand,
the C/T (rs5743836) polymorphism in the gene encoding Toll-like receptor 9,
the −81 C/T (rs 2273953) polymorphism in the 5′ UTR of the gene encoding Tumor protein P73,

or one or more polymorphisms in linkage disequilibrium with one or more of said polymorphisms.

More preferably, said intervention is a CT scan for lung cancer.

Still more preferably, the method is as described herein with reference to the examples and/or figures.

In a further aspect, the present invention provides a kit for assessing a subject's risk of developing lung cancer, said kit comprising a means of analysing a sample from said subject for the presence or absence of one or more polymorphisms disclosed herein.

### BRIEF DESCRIPTION OF FIGURES

FIG. 1: depicts a graph showing the likelihood of having lung cancer plotted against the SNP score derived from the 5 SNP panel shown in Table 16 herein.

FIG. 2: depicts a graph showing the log odds of having lung cancer plotted against the SNP score derived from the 5 SNP panel shown in Table 16 herein.

FIG. 3 depicts a graph showing the likelihood of having lung cancer plotted against the SNP score derived from an 11 SNP panel (11 SNP panel A) comprising SNPs 1-11 in Table 18 herein.

FIG. 4 depicts a receiver-operator curve analysis of sensitivity and specificity for the 11 SNP panel A.

FIG. 5 depicts a graph showing the distribution of frequencies of control smokers and lung cancer subjects plotted against SNP score derived from the 11 SNP panel A.

FIG. 6 depicts a graph showing the likelihood of having lung cancer plotted against the SNP score derived from a 16 SNP panel comprising SNPs 1-16 in Table 18 herein.

FIG. 7 depicts a receiver-operator curve analysis of sensitivity and specificity for the 16 SNP panel.

FIG. 8 depicts a graph showing the distribution of frequencies of control smokers and lung cancer subjects plotted against SNP score derived from the 16 SNP panel.

FIG. 9 depicts a graph showing the log odds of having lung cancer plotted against the SNP score derived from the 9 SNP panel described herein.

FIG. 10 depicts a receiver-operator curve analysis of sensitivity and specificity for the 9 SNP panel.

FIG. 11 depicts a graph showing the distribution of frequencies of control smokers and lung cancer subjects plotted against SNP score derived from the 9 SNP panel.

FIG. 12 depicts a graph showing the likelihood of having one of the four common types of lung cancer plotted against the SNP score, as described in Example 5.

FIG. 13a depicts a graph showing the frequency of lung cancer plotted against the SNP score derived from the 19 SNP panel described in Example 6 herein.

FIG. 13b depicts a graph showing the odds ratio of lung cancer according to the SNP score derived from the 19 SNP panel described in Example 6 herein.

FIG. 14 depicts a graph showing the distribution of frequencies of control smokers and lung cancer subjects plotted against SNP score derived from the 19 SNP panel described in Example 6 herein.

FIG. 15 depicts a graph showing the distribution of frequencies of control smokers (continuous line) and lung cancer subjects divided according to having normal lung function (Lung Ca with % predFEV1>median (73%)=dashed line) or with low lung function (Lung Ca with % predFEV1<median=dotted line) plotted against SNP score derived from the 19 SNP panel described in Example 6 herein.

### DESCRIPTION OF THE PREFERRED EMBODIMENTS

Using case-control studies the frequencies of several genetic variants (polymorphisms) of candidate genes in smokers who have developed lung cancer and blood donor controls have been compared. The majority of these candidate genes have confirmed (or likely) functional effects on gene expression or protein function. Specifically the frequencies of polymorphisms between blood donor controls, resistant smokers and those with lung cancer (subdivided into those with early onset and those with normal onset) have been compared. The present invention demonstrates that there are both protective and susceptibility polymorphisms present in selected candidate genes of the patients tested.

In one embodiment described herein 8 susceptibility genetic polymorphisms and 6 protective genetic polymorphism are identified. These are as follows:

| Gene and SNP | rs number | Genotype | Phenotype | OR | P value |
|---|---|---|---|---|---|
| Cerberus 1 (Cer 1) R19W A/G | rs10115703 | AA/AG | susceptiblility | 1.7 | 0.02 |
| XRCC4 Ser307Ser G/T | rs1056503 | GG/GT | susceptiblility | 1.3 | 0.04 |
| BRCA2 K3326X A/T | rs11571833 | AT/TT | susceptiblility | 2.5 | 0.04 |
| Integrin alpha-11 V433M A/G | rs2306022 | AA | susceptiblility | 4.3 | 0.002 |
| CAMKK1 E375G T/C | rs7214723 | TT | protective | 0.76 | 0.13 |
| P73 | rs2273953 | CC | protective | 0.46 | <0.001 |
| CYP3A43 C74 delA | | AT/TT | susceptiblility | 1.74 | 0.05 |
| BCL2 | rs2279115 | AA | protective | 0.69 | 0.05 |
| ITGB3 | rs2317676 | AG/GG | protective | 0.57 | 0.02 |
| DAT1 | rs6413429 | GT/TT | susceptibility | 1.6 | 0.05 |
| TNFR1 | rs1139417 | AA | susceptibility | 1.5 | 0.02 |
| DRD2 | rs1799732 | CDel/DelDel | protective | 0.61 | 0.02 |
| FasL | rs763110 | TT | protective | 0.61 | 0.05 |
| TLR9 | rs5743836 | CC | susceptibility | 3.1 | 0.03 |

A susceptibility genetic polymorphism is one which, when present, is indicative of an increased risk of developing lung cancer. In contrast, a protective genetic polymorphism is one which, when present, is indicative of a reduced risk of developing lung cancer.

As used herein, the phrase “risk of developing lung cancer” means the likelihood that a subject to whom the risk applies will develop lung cancer, and includes predisposition to, and potential onset of the disease. Accordingly, the phrase “increased risk of developing lung cancer” means that a subject having such an increased risk possesses an hereditary inclination or tendency to develop lung cancer. This does not mean that such a person will actually develop lung cancer at any time, merely that he or she has a greater likelihood of developing lung cancer compared to the general population of individuals that either does not possess a polymorphism associated with increased lung cancer or does possess a polymorphism associated with decreased lung cancer risk. Subjects with an increased risk of developing lung cancer include those with a predisposition to lung cancer, such as a tendency or predilection regardless of their lung function at the time of assessment, for example, a subject who is genetically inclined to lung cancer but who has normal lung function, those at potential risk, including subjects with a tendency to mildly reduced lung function who are likely to go on to suffer lung cancer if they keep smoking, and subjects with potential onset of lung cancer, who have a tendency to poor lung function on spirometry etc., consistent with lung cancer at the time of assessment.

Similarly, the phrase “decreased risk of developing lung cancer” means that a subject having such a decreased risk possesses an hereditary disinclination or reduced tendency to develop lung cancer. This does not mean that such a person will not develop lung cancer at any time, merely that he or she has a decreased likelihood of developing lung cancer compared to the general population of individuals that either does possess one or more polymorphisms associated with increased lung cancer, or does not possess a polymorphism associated with decreased lung cancer.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and may be in linkage disequilibrium. A haplotype can be identified by patterns of polymorphisms such as SNPs. Similarly, the term “single nucleotide polymorphism” or “SNP” in the context of the present invention includes single base nucleotide subsitutions and short deletion and insertion polymorphisms.

A reduced or increased risk of a subject developing lung cancer may be diagnosed by analysing a sample from said subject for the presence of a polymorphism selected from the group consisting of:
R19W A/G (rs10115703) in the gene encoding Cerberus 1 (Cer 1);
Ser307Ser G/T (rs1056503) in the X-ray repair complementing defective repair in Chinese hamster cells 4 gene (XRCC4);
K3326X A/T (rs11571833) in the breast cancer 2 early onset gene (BRCA2);
V433M A/G (rs2306022) in the gene encoding Integrin alpha-11;
E375G T/C (rs7214723) in the gene encoding Calcium/calmodulin-dependent protein kinase kinase 1 (CAMKK1);
A/T c74delA in the gene encoding cytochrome P450 polypeptide CYP3A43 (CYP3A43);
A/C (rs2279115) in the gene encoding B-cell CLL/lymphoma 2 (BCL2);
A/G at +3100 in the 3′UTR (rs2317676) of the gene encoding Integrin beta 3 (ITGB3);
−3714 G/T (rs6413429) in the gene encoding Dopamine transporter 1 (DAT1);
A/G (rs1139417) in the gene encoding Tumor necrosis factor receptor 1 (TNFR1);
C/Del (rs1799732) in the gene encoding Dopamine receptor D2 (DRD2);
C/T (rs763110) in the gene encoding Fas ligand (FasL); or
C/T (rs5743836) in the gene encoding Toll-like receptor 9 (TLR9)
−81 C/T (rs 2273953) in the 5′ UTR of the gene encoding Tumor protein P73 (P73);

or one or more polymorphisms which are in linkage disequilibrium with any one or more of the above group.

These polymorphisms can also be analysed in combinations of two or more, or in combination with other polymorphisms indicative of a subject's risk of developing lung cancer inclusive of the remaining polymorphisms listed above.

Expressly contemplated are combinations of the above polymorphisms with polymorphisms as described in PCT International application PCT/NZ02/00106, published as WO 02/099134, or as described in PCT International application PCT/NZ2006/000125, published as WO2006/123955, or those polymorphisms recited herein in Table 18.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;

or one or more polymorphisms in linkage disequilibrium with any one or more of these polymorphisms.

In one embodiment of the methods and uses of the present invention each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
V433M A/G (rs2306022) in the gene encoding ITGA11;

or one or more polymorphisms in linkage disequilibrium with any one or more of these polymorphisms.

In one embodiment of the methods and uses of the present invention each of the following polymorphisms are selected:
Rsa 1 C/T (rs2031920) in the gene encoding CYP 2E1;
−133 G/C (rs360721) in the promoter of the gene encoding Interleukin-18;
−251 A/T (rs4073) in the gene encoding Interleukin-8;
−511 A/G (rs 16944) in the gene encoding Interleukin 1B;
V433M A/G (rs2306022) in the gene encoding ITGA11;
Arg 197 Gln A/G (rs 1799930) in the gene encoding N-acetylcysteine transferase 2;
Ala 15 Thr A/G (rs4934) in the gene encoding α1-antichymotrypsin;
R19W A/G in the gene encoding Cerberus 1 (rs 10115703);
−3714 G/T (rs6413429) in the gene encoding DAT1 (rs6413429);
A/G (rs1139417) in the gene encoding TNFR1;
C/T (rs5743836) in the gene encoding TLR9;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
−751 G/T (rs 13181) in the promoter of the gene encoding XPD;
Phe 257 Ser C/T (rs3087386) in the gene encoding REV1;
C/T (rs763110) in the gene encoding FasL;

or one or more polymorphisms in linkage disequilibrium with any one or more of these polymorphisms.

Assays which involve combinations of polymorphisms, including those amenable to high throughput, such as those utilising microarrays, are preferred.

Statistical analyses, particularly of the combined effects of these polymorphisms, show that the genetic analyses of the present invention can be used to determine the risk quotient of any smoker and in particular to identify smokers at greater risk of developing lung cancer. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of smokers and non-smokers, as described herein, it is possible to implicate certain proteins in the development of lung cancer and improve the ability to identify which smokers are at increased risk of developing lung cancer-related impaired lung function and lung cancer for predictive purposes.

The present results show for the first time that the minority of smokers who develop lung cancer do so because they have one or more of the susceptibility polymorphisms and few or none of the protective polymorphisms defined herein. It is thought that the presence of one or more suscetptible polymorphisms, together with the damaging irritant and oxidant effects of smoking, combine to make this group of smokers highly susceptible to developing lung cancer. Additional risk factors, such as familial history, age, weight, pack years, etc., will also have an impact on the risk profile of a subject, and can be assessed in combination with the genetic analyses described herein.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

It will be apparent that polymorphsisms in linkage disequilibrium with one or more other polymorphism associated with increased or decreased risk of developing lung cancer will also provide utility as biomarkers for risk of developing lung cancer. The data presented herein shows that the frequency for SNPs in linkage disequilibrium is very similar. Accordingly, these genetically linked SNPs can be utilized in combined polymorphism analyses to derive a level of risk comparable to that calculated from the original SNP.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented herein in Table 26.

It will also be apparent that frequently a variety of nomenclatures may exist for any given polymorphism or for any given gene. For example, the polymorphism Arg 312 Gln in the gene encoding superoxide dismutase 3 (SOD3) is believed to have been referred to variously as Arg 213 Gly, +760 G/C, and Arg 231 Gly (rs1799895). In another example, the gene referred to herein as the breast cancer 2 early onset gene is also variously referred to as BRCC2, Breast Cancer 2 Gene, Breast Cancer Type 2, Breast Cancer Type 2 Susceptibility Gene, Breast cancer type 2 susceptibility protein, FACD, FAD, FAD1, FANCB, FANCD1, and Hereditary Breast Cancer 2. When referring to a susceptibility or protective polymorphism as herein described, such alternative nomenclatures are also contemplated by the present invention.

The methods of the invention are primarily directed to the detection and identification of the above polymorphisms associated with lung cancer, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. Initially, this was no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation. These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags. A preferred example is the use of mass spectrometric determination of a nucleic acid sequence which comprises the polymorphisms of the invention, for example, as shown herein in the Examples. Such mass spectrometric methods are known to those skilled in the art, and the genotyping methods of the invention are amenable to adaptation for the mass spectrometric detection of the polymorphisms of the invention, for example, the polymorphisms of the invention as shown in Table 16 herein.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomlQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonomous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each incorporated by reference).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with lung cancer. Such risk factors include epidemiological risk factors associated with an increased risk of developing lung cancer. Such risk factors include, but are not limited to smoking and/or exposure to tobacco smoke, age, sex and familial history. These risk factors can be used to augment an analysis of one or more polymorphisms as herein described when assessing a subject's risk of developing lung cancer.

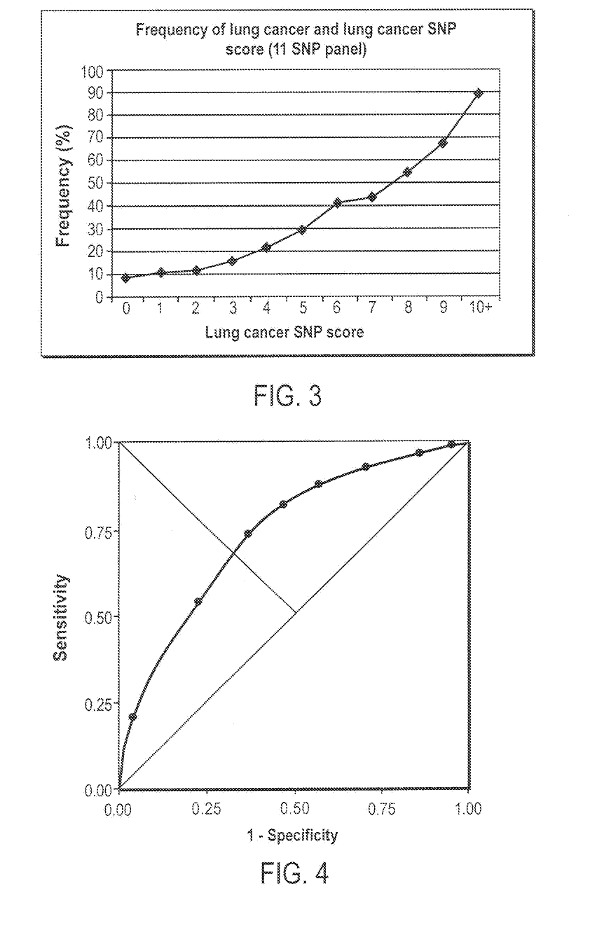

It is recognised that individual SNPs may confer weak risk of susceptibility or protection to a disease or phenotype of interest. These modest effects from individual SNPs are typically measured as odds ratios in the order of 1-3. The specific phenotype of interest may be a disease, such as lung cancer, or an intermediate phenotype based on a pathological, biochemical or physiological abnormality (for example, impaired lung function). As shown herein, when specific genotypes from individual SNPs are assigned a numerical value reflecting their phenotypic effect (for example, a positive value for susceptibility SNPs and a negative value for protective SNPs), the combined effects of these SNPs can be derived from an algorithm that calculates an overall score. Again as shown herein in a case-control study design, this SNP score is linearly related to the frequency of disease (or likelihood of having disease)—see for example FIGS. 3 and 4.

The SNP score provides a means of comparing people with different scores and their odds of having disease in a simple dose-response relationship. In this analysis, the people with the lowest SNP score are the referent group (Odds ratio=1) and those with greater SNP scores have a correspondingly greater odds (or likelihood) of having the disease—again in a linear fashion. The Applicants believe, without wishing to be bound by any theory, that the extent to which combining SNPs optimises these analyses is dependent, at least in part, on the strength of the effect of each SNP individually in a univariate analysis (independent effect) and/or multivariate analysis (effect after adjustment for effects of other SNPs or non-genetic factors) and the frequency of the genotype from that SNP (how common the SNP is). However, the effect of combining certain SNPs may also be in part related to the effect that those SNPs have on certain pathophysiological pathways that underlie the phenotype or disease of interest.

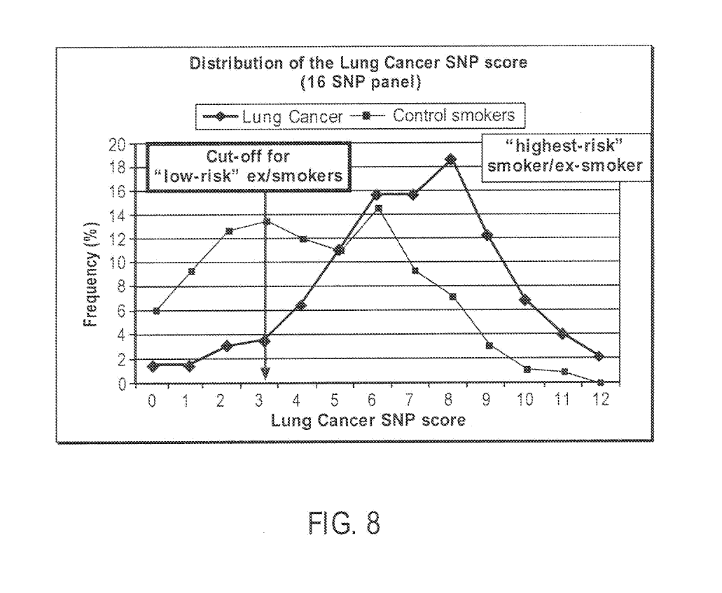

The Applicants have found that combining certain SNPs may increase the accuracy of the determination of risk or likelihood of disease in an unpredictable fashion. Specifically, when the distribution of SNP scores for the cases and controls are plotted according to their frequency, the ability to segment those with and without disease (or risk of disease) can be improved according to the specific combination of SNPs that are analysed. See, for example, the distributions for the 11 SNP panel A (FIG. 6) and for the 16 SNP panel (FIG. 8). It appears that this effect is not solely dependent on the number of relevant SNPs that are analysed in combination, nor the magnitude of their individual effects, nor their frequencies in the cases or controls. It further appears that the ability to improve this segmentation of the population into high and low risk is not due to any specific ratio of susceptibility or protective SNPs. The Applicants believe, without wishing to be bound by any theory, that the greater separation of the population in to high and low risk may at least partly be a function of identifying SNPs that confer a susceptibility or protective phenotype in important but independent pathophysiological pathways.

This observation has clinical utility in helping to define a threshold or cut-off level in the SNP score that will define a subgroup of the population to undergo an intervention. Such an intervention may be a diagnostic intervention, such as imaging test, other screening or diagnostic test (eg biochemical or RNA based test), or may be a therapeutic intervention, such as a chemopreventive therapy (for example, cisplatin or etoposide for small cell lung cancer), radiotherapy, or a preventive lifestyle modification (stopping smoking for lung cancer). In defining this clinical threshold, people can be prioritised to a particular intervention in such a way to minimise costs or minimise risks of that intervention (for example, the costs of image-based screening or expensive preventive treatment or risk from drug side-effects or risk from radiation exposure). In determining this threshold, one might aim to maximise the ability of the test to detect the majority of cases (maximise sensitivity) but also to minimise the number of people at low risk that require, or may be are otherwise eligible for, the intervention of interest.

Receiver-operator curve (ROC) analyses analyze the clinical performance of a test by examining the relationship between sensitivity and false positive rate (i.e., 1-specificity) for a single variable in a given population. In an ROC analysis, the test variable may be derived from combining several factors. Either way, this type of analysis does not consider the frequency distribution of the test variable (for example, the SNP score) in the population and therefore the number of people who would need to be screened in order to identify the majority of those at risk but minimise the number who need to be screened or treated. The Applicants have found that this frequency distribution plot may be dependent on the particular combination of SNPs under consideration and it appears it may not be predicted by the effect conferred by each SNP on its own nor from its performance characteristics (sensitivity and specificity) in an ROC analysis.

The data presented herein shows that determining a specific combination of SNPs can enhance the ability to segment or subgroup people into intervention and non-intervention groups in order to better prioritise these interventions. Such an approach is useful in identifying which smokers might be best prioritised for interventions, such as CT screening for lung cancer. Such an approach could also be used for initiating treatments or other screening or diagnostic tests. As will be appreciated, this has important cost implications to offering such interventions.

Accordingly, the present invention also provides a method of assessing a subject's suitability for an intervention diagnostic of or therapeutic for a disease, the method comprising:
a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;
and

b) providing a distribution of net scores for disease sufferers and non-sufferers wherein the net scores for disease sufferers and non-sufferers are or have been determined in the same manner as the net score determined for said subject;

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

The intervention may be a diagnostic test for the disease, such as a blood test or a CT scan for lung cancer. Alternatively, the intervention may be a therapy for the disease, such as chemotherapy or radiotherapy, including a preventative therapy for the disease, such as the provision of motivation to the subject to stop smoking.

As described herein, a distribution of SNP scores for lung cancer sufferers and resistant smoker controls (non-sufferers) can be established using the methods of the invention. For example, a distribution of SNP scores derived from the 16 SNP panel consisting of the protective and susceptibility polymorphisms selected from the group consisting of the −133 G/C polymorphism in the Interleukin-18 gene, the −1053 C/T polymorphism in the CYP 2E1 gene, the Arg197gln polymorphism in the Nat2 gene, the −511 G/A polymorphism in the Interleukin 1B gene, the Ala 9 Thr polymorphism in the Anti-chymotrypsin gene, the S allele polymorphism in the Alpha1-antitrypsin gene, the −251 A/T polymorphism in the Interleukin-8 gene, the Lys 751 gln polymorphism in the XPD gene, the +760 G/C polymorphism in the SOD3 gene, the Phe257Ser polymorphism in the REV gene, the Z alelle polymorphism in the Alpha1-antitrypsin gene, the R19W A/G polymorphism in the Cerberus 1 (Cer 1) gene, the Ser307Ser G/T polymorphism in the XRCC4 gene, the K3326X A/T polymorphism in the BRCA2 gene, the V433M A/G polymorphism in the Integrin alpha-11 gene, and the E375G T/C polymorphism in the CAMKK1 gene, among lung cancer sufferers and non-sufferers is described herein. As shown herein, a threshold SNP score can be determined that separates people into intervention and non-intervention groups, so as to better prioritise those individuals suitable for such interventions.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to lung cancer also has application in the design and/or screening of candidate therapeutics. This is particularly the case where the association between a susceptibility or protective polymorphism is manifested by either an upregulation or downregulation of expression of a gene. In such instances, the effect of a candidate therapeutic on such upregulation or downregulation is readily detectable.

For example, in one embodiment existing human lung organ and cell cultures are screened for polymorphisms as set forth above. (For information on human lung organ and cell cultures, see, e.g.: Bohinski et al. (1996) Molecular and Cellular Biology 14:5671-5681; Collettsolberg et al. (1996) Pediatric Research 39:504; Hermanns et al. (2004) Laboratory Investigation 84:736-752; Hume et al. (1996) In Vitro Cellular & Developmental Biology-Animal 32:24-29; Leonardi et al. (1995) 38:352-355; Notingher et al. (2003) Biopolymers (Biospectroscopy) 72:230-240; Ohga et al. (1996) Biochemical and Biophysical Research Communications 228:391-396; each of which is hereby incorporated by reference in its entirety.) Cultures representing susceptibility and protective genotype groups are selected, together with cultures which are putatively “normal” in terms of the expression of a gene which is either upregulated or downregulated where a protective polymorphism is present.

Samples of such cultures are exposed to a library of candidate therapeutic compounds and screened for any or all of: (a) downregulation of susceptibility genes that are normally upregulated in susceptibility polymorphisms; (b) upregulation of susceptibility genes that are normally downregulated in susceptibility polymorphisms; (c) downregulation of protective genes that are normally downregulated or not expressed (or null forms are expressed) in protective polymorphisms; and (d) upregulation of protective genes that are normally upregulated in protective polymorphisms. Compounds are selected for their ability to alter the regulation and/or action of susceptibility genes and/or protective genes in a culture having a susceptibility polymorphisms.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

The methods of the invention are primarily directed at assessing risk of developing lung cancer. Lung cancer can be divided into two main types based on histology—non-small cell (approximately 80% of lung cancer cases) and small-cell (roughly 20% of cases) lung cancer. This histological division also reflects treatment strategies and prognosis.

The non-small cell lung cancers (NSCLC) are generally considered collectively because their prognosis and management is roughly identical. For non-small cell lung cancer, prognosis is poor. The most common types of NSCLC are adenocarcinoma, which accounts for 50% to 60% of NSCLC, squamous cell carcinoma, and large cell carcinoma.

Adenocarcinoma typically originates near the gas-exchanging surface of the lung. Most cases of the adenocarcinoma are associated with smoking. However, adenocarcinoma is the most common form of lung cancer among non-smokers. A subtype of adenocarcinoma, the bronchioalveolar carcinoma, is more common in female non-smokers.

Squamous cell carcinoma, accounting for 20% to 25% of NSCLC, generally originates in the larger breathing tubes. This is a slower growing form of NSCLC.

Large cell carcinoma is a fast-growing form that grows near the surface of the lung. An initial diagnosis of large cell carcinoma is frequently reclassified to squamous cell carcinoma or adenocarcinoma on further investigation.

For small cell lung cancer (SCLC), prognosis is also poor. It tends to start in the larger breathing tubes and grows rapidly becoming quite large. It is initially more sensitive to chemotherapy, but ultimately carries a worse prognosis and is often metastatic at presentation. SCLC is strongly associated with smoking.

Other types of lung cancer include carcinoid lung cancer, adenoid cystic carcinoma, cylindroma, mucoepidermoid carcinoma, and metastatic cancers which originate in other parts of the body and metatisize to the lungs. Generally, these cancers are identified by the site of origin, i.e., a breast cancer metastasis to the lung is still known as breast cancer. Conversely, the adrenal glands, liver, brain, and bone are the most common sites of metastasis from primary lung cancer itself.

Due to the poor prognosis for lung cancer sufferors, early detection is of paramount importance. However, the screening methodologies currently widely available have been reported to be largely ineffective. Regular chest radiography and sputum examination programs were not effective in reducing mortality from lung cancer, leading the authors to conclude that the current evidence did not support screening for lung cancer with chest radiography or sputum cytology, and that frequent chest x-ray screening might be harmful. (See Manser R L, et al., Screening for lung cancer. Cochrane Database of Systematic Reviews 2004, Issue 1. Art. No.: CD001991. DOI: 10.1002/14651858.CD001991.pub2.).

Computed tomography (CT) scans can uncover tumors not yet visible on an X-ray. CT scanning is now being actively evaluated as a screening tool for lung cancer in high risk patients. In a study of over 31,000 high-risk patients, 85% of the 484 detected lung cancers were stage I and were considered highly treatable (see Henschke C I, et al., Survival of patients with stage I lung cancer detected on CT screening. N Engl J Med., 355(17):1763-71, (2006).

In contrast, a recent study in which 3,200 current or former smokers were screened for 4 years and offered 3 or 4 CT scans reported increased diagnoses of lung cancer and increased surgeries, but no significant differences between observed and expected numbers of advanced cancers or deaths (see Bach P B, et al., Computed Tomography Screening and Lung Cancer Outcomes, JAMA., 297:953-961 (2007)).

It should be noted that screening studies have only been done in high risk populations, such as smokers and workers with occupational exposure to certain substances. A more definitive appraisal of the efficacy of screening using CT may need await the results of ongoing randomized trials in the U.S. and Europe. This is important when one considers that repeated radiation exposure from screening could actually induce carcinogenesis in a small percentage of screened subjects, so this risk should be mitigated by a (relatively) high prevalence of lung cancer in the population being screened. This high prevalence can be achieved by prescreening prior to CT scanning by, for example, the methods described herein.

The invention will now be described in more detail, with reference to the following non-limiting examples.

### Example 1

### Case Association Study

### Introduction

Case-control association studies allow the careful selection of a control group where matching for important risk factors is critical. In this study, smokers diagnosed with lung cancer and smokers without lung cancer with normal lung function were compared. This unique control group is highly relevant as it is impossible to pre-select smokers with zero risk of lung cancer—i.e., those who although smokers will never develop lung cancer. Smokers with a high pack year history and normal lung function were used as a “low risk” group of smokers, as the Applicants believe it is not possible with current knowledge to identify a lower risk group of smokers. The Applicants believe, without wishing to be bound by any theory, that this approach allows for a more rigorous comparison of low penetrant, high frequency polymorphisms that may confer an increased risk of developing lung cancer. The Applicants also believe, again without wishing to be bound by any theory, that there may be polymorphisms that confer a degree of protection from lung cancer which may only be evident if a smoking cohort with normal lung function is utilised as a comparator group. Thus smokers with lung cancer would be expected to have a lower frequency of these polymorphisms compared to smokers with normal lung function and no diagnosed lung cancer.

### Methods

### Subject Recruitment

Subjects of European decent who had smoked a minimum of fifteen pack years and diagnosed with lung cancer were recruited. Subjects met the following criteria: diagnosed with lung cancer based on radiological and histological grounds, including primary lung cancers with histological types of small cell lung cancer, squamous cell lung cancer, adenocarinoma of the lung, non-small cell cancer (where histological markers can not distinguish the subtype) and broncho-alveolar carcinoma. Subjects could be of any age and at any stage of treatment after the diagnosis had been confirmed. 239 subjects were recruited, of these 53% were male, the mean FEV1/FVC (1SD) was 61% (14), mean FEV1 as a percentage of predicted was 71 (22). Mean age, cigarettes per day and pack year history was 69 yrs (11), 18 cigarettes/day (11) and 38 pack years (31), respectively. 484 European subjects who had smoked a minimum of twenty pack years and who had never suffered breathlessness and had not been diagnosed with an obstructive lung disease or lung cancer in the past were also studied. This control group was recruited through clubs for the elderly and consisted of 60% male, the mean FEV1/FVC (1SD) was 76% (8), mean FEV1 as a percentage of predicted was 101 (10). Mean age, cigarettes per day and pack year history was 60 yrs (12), 24 cigarettes/day (12) and 41 pack years (25), respectively. Using a PCR based method (Sandford et al., 1999), all subjects were genotyped for the cd-antitrypsin mutations (S and Z alleles) and those with the ZZ allele were excluded. On regression analysis, the age difference and pack years difference observed between lung cancer sufferers and resistant smokers was found not to determine FEV or lung cancer.

This study shows that polymorphisms found in greater frequency in lung cancer patients compared to resistant smokers may reflect an increased susceptibility to the development of lung cancer. Similarly, polymorphisms found in greater frequency in resistant smokers compared to lung cancer may reflect a protective role.

### Summary of Characteristics for the Lung Cancer Subjects and Resistant Smokers.

| Parameter: | Lung Cancer | Resistant smokers | |
|---|---|---|---|
| Mean (1 SD) | N = 239 | N = 484 | Differences |
| % male | 53% | 60% | ns |
| Age (yrs) | 69 ( 11 ) | 60 (12) | P < 0.05 |
| Pack years | 38 ( 31 ) | 41 (25) | P < 0.05 |
| Cigarettes/day | 18 ( 11 ) | 24(12) | ns |
| FEV1 (L) | 1.8 (0.6) | 2.8 (0.7) | P < 0.05 |
| FEV1 % predict | 71 ( 22 ) | 101% (10) | P < 0.05 |
| FEV1/FVC | 61 ( 14 ) | 76 (8) | P < 0.05 |

Means and 1 SD

### Polymorphism Genotyping Using the Sequenom Autoflex Mass Spectrometer

Genomic DNA was extracted from whole blood samples (Maniatis, T., Fritsch, E. F. and Sambrook, J., Molecular Cloning Manual. 1989). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10×Buffer 15 mM MgCl2 1.25×, 25 mM MgCl2 1.625 mM, dNTP mix 25 mM 500 uM, primers 4 uM 100 nM, Taq polymerase (Quiagen hot start) 0.15 U/reaction, Genomic DNA 10 ng/ul. Cycling times were 95° C. for 15 mM, (5° C. for 15 s, 56° C. 30 s, 72° C. 30 s for 45 cycles with a prolonged extension time of 3 min to finish. We used shrimp alkaline phosphotase (SAP) treatment (2 ul to 5 ul per PCR reaction) incubated at 35° C. for 30 min and extension reaction (add 2 ul to 7 ul after SAP treatment) with the following volumes per reaction of: water, 0.76 ul; hME 10× termination buffer, 0.2 ul; hME primer (10 uM), 1 ul; MassEXTEND enzyme, 0.04 ul.

TABLE 1
Sequenom conditions for genotyping

| SNP_ID | 2nd-PCRP | 1st-PCRP |
|---|---|---|
| rs11571833 | ACGTTGGATGCTGAATTCTCCTCAGATGAC | ACGTTGGATGAATGCAAGTTCTTCGTCAGC |
| | [SEQ.ID.NO. 1] | [SEQ.ID.NO. 2] |
| rs7214723 | ACGTTGGATGAAAACTCAGACACCAGGAGC | ACGTTGGATGAGATCAAGAATGAGCCCGTG |
| | [SEQ.ID.NO. 3] | [SEQ.ID.NO. 4] |
| rs10115703 | ACGTTGGATGCCTCTTATTTCAGCTGCTGG | ACGTTGGATGAGAGAACTCTGATTCTGGCG |
| | [SEQ.ID.NO. 5] | [SEQ.ID.NO. 6] |
| rs2306022 | ACGTTGGATGACCTTGCCCGTGTGGTTGAA | ACGTTGGATGTGGCAGGGTACACAGTCACA |
| | [SEQ.ID.NO. 7] | [SEQ.ID.NO. 8] |
| rs1056503 | ACGTTGGATGCTGCTGTTTCTCAGAGTTTC | ACGTTGGATGGCCTGATTCTTCACTACCTG |
| | [SEQ.ID.NO. 9] | [SEQ.ID.NO. 10] |
| rs2273953 | ACGTTGGATGTGCTCAGGTGTCATTCCTTC | ACGTTGGATGGGTGGACTGGGCCATCTTC |
| | [SEQ.ID.NO. 26] | [SEQ.ID.NO. 27] |
| c74delA | ACGTTGGATGTTCTGTAACCTGGCTTTCTC | ACGTTGGATGCCAGGAATTCCCAGCTTCTT |
| | [SEQ.ID.NO. 28] | [SEQ.ID.NO. 29] |
| rs1799732 | ACGTTGGATGCAAAACAAGGGATGGCGGAA | ACGTTGGATGAAAGGAGCTGTACCTCCTCG |
| | [SEQ.ID.NO. 30] | [SEQ.ID.NO. 31] |
| rs2279115 | ACGTTGGATGATCAGAAGAGGATTCCTGCC | ACGTTGGATGTTCACGCCTCCCCAGGAGA |
| | [SEQ.ID.NO. 32] | [SEQ.ID.NO. 33] |
| rs2317676 | ACGTTGGATGTATGAACTGGGAGATGCTGG | ACGTTGGATGTGTTGGGAGTGAGGATGTCT |
| | [SEQ.ID.NO. 34] | [SEQ.ID.NO. 35] |
| rs5743836 | ACGTTGGATGTTGGGATGTGCTGTTCCCTC | ACGTTGGATGAGCAGAGACATAATGGAGGC |
| | [SEQ.ID.NO. 36] | [SEQ.ID.NO. 37] |
| rs6413429 | ACGTTGGATGTGTCAGGAGGCCTTCAGGTG | ACGTTGGATGGTTTTATGAGGGCACTGGTC |
| | [SEQ.ID.NO. 38] | [SEQ.ID.NO. 39] |
| rs1139417 | ACGTTGGATGAGGCCATAGCTGTCTGGCAT | ACGTTGGATGTTCCCTTTGTCCCTGGTCT |
| | [SEQ.ID.NO. 40] | [SEQ.ID.NO. 41] |
| rs763110 | ACGTTGGATGAGGCTGCAAACCAGTGGAAC | ACGTTGGATGCTGGGCAAACAATGAAAATG |
| | [SEQ.ID.NO. 42] | [SEQ.ID.NO. 43] |

| SNP_ID | AMP_LEN | UP_CONF | MP_CONF | Tm(NN) | PcGC | PWARN | UEP_DIR | UEP_MASS |
|---|---|---|---|---|---|---|---|---|
| rs11571833 | 109 | 96.8 | 69.1 | 46.3 | 44.4 | | F | 5409.5 |
| rs7214723 | 113 | 99.3 | 69.1 | 61.3 | 58.3 | dH | F | 7304.7 |
| rs10115703 | 101 | 98.7 | 69.1 | 59 | 50 | | R | 7884.1 |
| rs2306022 | 111 | 91.8 | 90.9 | 53.8 | 68.8 | D | R | 4867.2 |
| rs1056503 | 104 | 98.5 | 90.9 | 48 | 42.1 | | R | 5775.8 |
| rs2273953 | 98 | 90.6 | 90.6 | 49.3 | 58.8 | H | R | 5137.3 |
| c74delA | 101 | 94.9 | 69.7 | 45.7 | 25 | D | F | 7295.8 |
| rs1799732 | 99 | 97.3 | 66.7 | 59.5 | 66.7 | d | F | 6183 |
| rs2279115 | 99 | 88.3 | 78.5 | 52.3 | 64.7 | d | F | 5073.3 |
| rs2317676 | 97 | 98.7 | 66.7 | 63.3 | 62.5 | DH | R | 7298.7 |
| rs5743836 | 100 | 98.6 | 88.1 | 53 | 64.7 | | R | 5104.3 |
| rs6413429 | 93 | 94.2 | 66.7 | 56.5 | 70.6 | D | F | 5196.4 |
| rs1139417 | 99 | 92.2 | 99.6 | 56.2 | 70.6 | d | F | 5098.3 |
| rs763110 | 92 | 92.8 | 66.7 | 56.3 | 44 d | | R | 7591.9 |

| | | EXT1_ | EXT1_ | |
|---|---|---|---|---|
| SNP_ID | UEP_SEQ | CALL | MASS | EXT1_SEQ |
| rs11571833 | CCTCAGATGACTCCATTT | A | 5680.7 | CCTCAGATGACTCCATTTA |
| | [SEQ.ID.NO. 11] | | | [SEQ.ID.NO. 12] |
| rs7214723 | TGTTCCCCTGGGTGGACAACTCAC | C | 7551.9 | TGTTCCCCTGGGTGGACAACTCACC |
| | [SEQ.ID.NO. 13] | | | [SEQ.ID.NO. 14] |
| rs10115703 | TACTCCTGCCTCTAGGAAAGACCACA | G | 8131.3 | TACTCCTGCCTCTAGGAAAGACCACAC |
| | [SEQ.ID.NO. 15] | | | [SEQ.ID.NO. 16] |
| rs2306022 | CCCTGCCTGGAGGACA | G | 5114.4 | CCCTGCCTGGAGGACAC |
| | [SEQ.ID.NO. 17] | | | [SEQ.ID.NO. 18] |
| rs1056503 | CTGAGATGTGCTCCTTTTT | G | 6022.9 | CTGAGATGTGCTCCTTTTTC |
| | [SEQ. ID.NO. 19] | | | [SEQ.ID.NO. 20] |
| rs2273953 | CTTCCTTCCTGCAGAGG | T | 5408.6 | CTTCCTTCCTGCAGAGGA |
| | [SEQ.ID.NO. 44] | | | [SEQ.ID.NO. 45] |
| c74delA | GGCTTTCTCTTTTATTTTATAGTT | C | 7542.9 | GGCTTTCTCTTTTATTTTATAGTTC |
| | [SEQ.ID.NO. 46] | | | [SEQ.ID.NO. 47] |
| rs1799732 | CCCAACCCCTCCTACCCGTTC | C | 6430.2 | CCCAACCCCTCCTACCCGTTCC |
| | [SEQ.ID.NO. 48] | | | [SEQ.ID.NO. 49] |
| rs2279115 | GGCTCCTTCATCGTCCC | C | 5320.5 | GGCTCCTTCATCGTCCCC |
| | [SEQ.ID.NO. 50] | | | [SEQ.ID.NO. 51] |
| rs2317676 | GATGCTGGTACATCCCCCAGGCCA | G | 7545.9 | GATGCTGGTACATCCCCCAGGCCAC |
| | [SEQ.ID.NO. 52] | | | [SEQ.ID.NO. 53] |
| rs5743836 | GCTGTTCCCTCTGCCTG | T | 5375.5 | GCTGTTCCCTCTGCCTGA |
| | [SEQ.ID.NO. 54] | | | [SEQ.ID.NO. 55] |
| rs6413429 | GGAGGGCTCCACCCTGA | G | 5483.6 | GGAGGGCTCCACCCTGAG |
| | [SEQ.ID.NO. 56] | | | [SEQ.ID.NO. 57] |
| rs1139417 | CCTGACCTGCTGCTGCC | A | 5369.5 | CCTGACCTGCTGCTGCCA |
| | [SEQ.ID.NO. 58] | | | [SEQ.ID.NO. 59] |
| rs763110 | AACCCACAGAGCTGCTTTGTATTTC | T | 7863.2 | AACCCACAGAGCTGCTTTGTATTTCA |
| | [SEQ.ID.NO. 60] | | | [SEQ.ID.NO. 61] |

| | EXT2 | EXT2 | |
|---|---|---|---|
| SNP_ID | CALL | MASS | EXT2_SEQ |
| rs11571833 | T | 5736.6 | CCTCAGATGACTCCATTTT [SEQ.ID.NO. 21] |
| rs7214723 | T | 7631.8 | TGTTCCCCTGGGTGGACAACTCACT [SEQ.ID.NO. 22] |
| rs10115703 | A | 8211.2 | TACTCCTGCCTCTAGGAAAGACCACAT [SEQ.ID.NO. 23] |
| rs2306022 | A | 5194.3 | CCCTGCCTGGAGGACAT [SEQ.ID.NO. 24] |
| rs1056503 | T | 6047 | CTGAGATGTGCTCCTTTTTA [SEQ.ID.NO. 25] |
| rs2273953 | C | 5424.6 | CTTCCTTCCTGCAGAGGG [SEQ.ID.NO. 62] |
| c74delA | A | 7567 | GGCTTTCTCTTTTATTTTATAGTTA [SEQ.ID.NO. 63] |
| rs1799732 | DEL | 6454.2 | CCCAACCCCTCCTACCCGTTCA [SEQ.ID.NO. 64] |
| rs2279115 | A | 5344.5 | GGCTCCTTCATCGTCCCA [SEQ.ID.NO. 65] |
| rs2317676 | A | 7625.8 | GATGCTGGTACATCCCCCAGGCCAT [SEQ.ID.NO. 66] |
| rs5743836 | C | 5391.5 | GCTGTTCCCTCTGCCTGG [SEQ.ID.NO. 67] |
| rs6413429 | T | 5523.5 | GGAGGGCTCCACCCTGAT [SEQ.ID.NO. 68] |
| rs1139417 | G | 5385.5 | CCTGACCTGCTGCTGCCG [SEQ.ID.NO. 69] |
| rs763110 | C | 7879.2 | AACCCACAGAGCTGCTTTGTATTTCG [SEQ.ID.NO. 70] |
| | EXT3 | EXT3 | |
| SNP_ID | CALL | MASS | EXT3_SEQ |
| c74delA | G | 7583 | GGCTTTCTCTTTTATTTTATAGTTG [SEQ.ID.NO. 71] |
| | EXT4 | EXT4 | |
| SNP_ID | CALL | MASS | EXT4_SEQ |
| c74delA | T | 7622.8 | GGCTTTCTCTTTATTTTATAGTTT [SEQ.ID.NO. 72] |

### Results

### Univariate Analyses:

TABLE 2
Cerberus 1 (Cer 1) R19W A/G (rs 10115703) polymorphism allele and
genotype frequencies in the Lung cancer patients and resistant smokers.

| | Allele* | | Genotype | | |
|---|---|---|---|---|---|
| Frequency | A | G | AA | AG | GG |
| Lung Cancer | 47 (10%) | 421 (90%) | 2 (1%) | 43 (18%) | 189 (81%) |
| n = 234 (%) | | | | | |
| Resistant | 66 (7%) | 878 (93%) | 7 (1%) | 52 (11%) | 413 (88%) |
| n = 472 (%) | | | | | |

*number of chromosomes (2n)

Genotype. AA/AG vs GG for lung cancer vs resistant, Odds ratio (OR)=1.7, 95% confidence limits 1.1-2.6, χ² (Yates uncorrected)=5.63, p=0.02,

AA/AG genotype=susceptibility (GG protective)

Allele. A vs G for lung cancer vs resistant, Odds ratio (OR)=1.5, 95% confidence limits 1.0-2.2, χ² (Yates uncorrected)=3.95, p=0.05,

A allele=susceptibility

TABLE 3
XRCC4 Ser307Ser G/T (rs1056503) polymorphism allele and genotype
frequencies in the Lung cancer patients and resistant smokers.

| | Allele* | | Genotype | | |
|---|---|---|---|---|---|
| Frequency | G | T | GG | GT | TT |
| Lung Cancer | 68 (15%) | 374 (85%) | 8 (4%) | 52 (24%) | 161 (72%) |
| n = 221 (%) | | | | | |
| Resistant | 66 (11%) | 838 (89%) | 5 (1%) | 98 (21%) | 370 (78%) |
| n = 473 (%) | | | | | |

*number of chromosomes (2n)

Genotype. GG/GT vs TT for lung cancer vs resistant, Odds ratio (OR)=1.3, 95% confidence limits 0.9-2.0, χ² (Yates uncorrected)=2.4, p=0.12,

GG/GT genotype=susceptibility (TT protective)

Allele. G vs T for lung cancer vs resistant, Odds ratio (OR)=1.4, 95% confidence limits 1.0-2.0, χ² (Yates uncorrected)=4.28, p=0.04,

G allele=susceptibility

TABLE 4
BRCA2 K3326X A/T (rs 11571833) polymorphism allele and genotype
frequencies in the Lung cancer patients and resistant smokers.

| | Allele* | | Genotype | | |
|---|---|---|---|---|---|
| Frequency | A | T | AA | AT | TT |
| Lung Cancer | 450 (97%) | 12 (3%) | 220 (95%) | 10 (4%) | 1 (0.4%) |
| n = 231 (%) | | | | | |
| Resistant | 915 (99%) | 9 (1%) | 453 (98%) | 9 (2%) | 0 (0%) |
| n = 462 (%) | | | | | |

*number of chromosomes (2n)

Genotype. AT/TT vs AA for lung cancer vs resistant, Odds ratio (OR)=2.5, 95% confidence limits 1.0-6.7, χ² (Yates uncorrected)=4.34, p=0.04,

AT/TT genotype=susceptibility (AA protective)

Allele. T vs A for lung cancer vs resistant, Odds ratio (OR)=2.7, 95% confidence limits 1.1-7.0, χ² (Yates uncorrected)=5.44, p=0.02,

T allele=susceptibility

TABLE 5
Integrin alpha-11 V433M A/G (rs 2306022) polymorphism allele and
genotype frequencies in the Lung cancer patients and resistant smokers.

| | Allele* | | Genotype | | |
|---|---|---|---|---|---|
| Frequency | A | G | AA | AG | GG |
| Lung Cancer | 60 (13%) | 406 (87%) | 12 (5%) | 36 (15%) | 185 (79%) |
| n = 233 (%) | | | | | |
| Resistant | 89 (9%) | 863 (91%) | 6 (1%) | 77 (16%) | 393 (83%) |
| n = 476 (%) | | | | | |

*number of chromosomes (2n)

Genotype. AA vs AG/GG for lung cancer vs resistant, Odds ratio (OR)=4.3, 95% confidence limits 1.5-12.9, χ² (Yates uncorrected)=9.55, p=0.002,

AA genotype=susceptibility

Allele. A vs G for lung cancer vs resistant, Odds ratio (OR)=1.4, 95% confidence limits 1.0-2.1, χ² (Yates uncorrected)=4.14, p=0.04,

A allele=susceptibility

TABLE 6
CAMKK1 Calcium/calmodulin-dependent protein kinase kinase 1
E375G T/C (rs7214723) polymorphism allele and genotype frequencies
in the Lung cancer patients and resistant smokers.

| | Allele* | | Genotype | | |
|---|---|---|---|---|---|
| Frequency | T | C | TT | TC | CC |
| Lung Cancer | 239 (51%) | 227 (49%) | 62 (26%) | 115 (49%) | 56 (24%) |
| n = 233 (%) | | | | | |
| Resistant | 514 (56%) | 412 (44%) | 149 (32%) | 216 (47%) | 98 (21%) |
| n = 463 (%) | | | | | |

*number of chromosomes (2n)

Genotype. TT vs TC/CC for lung cancer vs resistant, Odds ratio (OR)=0.76, 95% confidence limits 0.5-1.1, χ² (Yates uncorrected)=2.27, p=0.13,

TT genotype=protective

Allele. T vs C for lung cancer vs resistant, Odds ratio (OR)=0.84, 95% confidence limits 0.7-1.1, χ² (Yates uncorrected)=2.22, p=0.14,

T allele=protective

TABLE 7
P73 C/T (rs 2273953) polymorphism allele and genotype frequencies in
the Lung cancer patients and resistant smokers.

| | Allele* | | Genotype | | |
|---|---|---|---|---|---|
| Frequency | C | T | CC | CT | TT |
| Lung Cancer | 316 (69%) | 142 (31%) | 99 (43%) | 118 (52%) | 12 (5%) |
| n = 229 (%) | | | | | |
| Resistant | 742 (78%) | 206 (22%) | 295 (62%) | 152 (32%) | 27 (6%) |
| n = 474 (%) | | | | | |

*number of chromosomes (2n)

Genotype. CC vs CT/TT for lung cancer vs resistant, Odds ratio (OR)=0.46, 95% confidence limits 0.33-0.64, χ2 (Yates uncorrected)=22.0, p<0.001,

CC genotype=protective (CT/TT susceptible)

Allele. C vs T for lung cancer vs resistant, Odds ratio (OR)=0.62, 95% confidence limits 0.48-0.80, χ2 (Yates corrected)=14.0, p<0.001,

C allele=protective

TABLE 8
CYP 3A43 A/T c74delA polymorphism allele and genotype frequencies in
the Lung cancer patients and resistant smokers.

| | Allele* | | Genotype | | |
|---|---|---|---|---|---|
| Frequency | A | T | AA | AT | TT |
| Lung Cancer | 442 (94%) | 26 (6%) | 209 (89%) | 24 (10%) | 1 (0.5%) |
| n = 234 (%) | | | | | |
| Resistant | 935 (97%) | 31 (3%) | 452 (94%) | 31 (6%) | 0 (0%) |
| n = 483 (%) | | | | | |

*number of chromosomes (2n)

Genotype. AT/TT vs AA for lung cancer vs resistant, Odds ratio (OR)=1.74, 95% confidence limits 0.97-3.13, χ2=(Yates uncorrected)=4.0, p=0.05,

AT/TT genotype=susceptible

Allele. T vs A for lung cancer vs resistant, Odds ratio (OR)=1.8, 95% confidence limits 1-3.1, χ2 (Yates uncorrected)=4.54, p=0.03,

T allele=susceptible

TABLE 9
BCL2 A/C (rs 2279115) polymorphism allele and genotype frequencies in
the Lung cancer patients and resistant smokers.

| | Allele* | | Genotype | | |
|---|---|---|---|---|---|
| Frequency | A | C | AA | AC | CC |
| Lung Cancer | 223 (47%) | 249 (53%) | 55 (23%) | 113 (48%) | 68 (29%) |
| n = 236 (%) | | | | | |
| Resistant | 513 (54%) | 445 (46%) | 146 (31%) | 221 (46%) | 112 (23%) |
| n = 479 (%) | | | | | |

*number of chromosomes (2n)

Genotype. AA vs AC/CC for lung cancer vs resistant, Odds ratio (OR)=0.69, 95% confidence limits 0.48-1.0, χ2 (Yates uncorrected)=4.0, p=0.05,

AA genotype=protective

Allele. A vs C for lung cancer vs resistant, Odds ratio (OR)=0.78, 95% confidence limits 0.62-0.97, χ2 (Yates corrected)=5.0, p=0.02,

A allele=protective

TABLE 10
ITGB3 A/G (rs 2317676) polymorphism allele and genotype
frequencies in the Lung cancer patients and resistant smokers.

| | Allele* | | Genotype | | |
|---|---|---|---|---|---|
| Frequency | A | G | AA | AG | GG |
| Lung Cancer | 445 | 23 (5%) | 211 | 23 (10%) | 0 (0%) |
| n = 234 (%) | (95%) | | (90%) | | |
| Resistant | 884 | 84 (9%) | 406 | 72 (15%) | 6 (1%) |
| n = 484 (%) | (91%) | | (84%) | | |

*number of chromosomes (2n)

Genotype. AG/GG vs AA for lung cancer vs resistant, Odds ratio (OR)=0.57, 95% confidence limits 0.34-0.95, χ2 (Yates uncorrected)=5.2, p=0.02,

AG/GG genotype=protective

Allele. G vs A for lung cancer vs resistant, Odds ratio (OR)=0.54, 95% confidence limits 0.33-0.89, χ2 (Yates uncorrected)=6.5, p=0.01,

G allele=protective

Integrin beta 3 is also referred to as platelet glycoprotein Ma or antigen CD61.

TABLE 11
DAT1 G/T (rs 6413429) polymorphism allele and genotype frequencies
in the Lung cancer patients and resistant smokers.

| | Allele* | | Genotype | | |
|---|---|---|---|---|---|
| Frequency | G | T | GG | GT | TT |
| Lung Cancer | 427 (92%) | 37 (8%) | 195 (84%) | 37 (16%) | 0 (0%) |
| n = 232 (%) | | | | | |
| Resistant | 914 (94%) | 56 (6%) | 433 (89%) | 48 (10%) | 4 (1%) |
| n = 485 (%) | | | | | |

*number of chromosomes (2n)

Genotype. TT/GT vs GG for lung cancer vs resistant, Odds ratio (OR)=1.6, 95% confidence limits 1.0-2.6, χ2 (Yates uncorrected)=3.9, p=0.05,

TT/GT genotype=susceptible

Dopamine transporter 1 (DAT1) is also known as solute carrier family 6 (neurotransmitter transporter, dopamine), member 3 (SLC6A3).

TABLE 12
TNFR1 A/G (rs1139417) polymorphism allele and genotype frequencies
in the Lung cancer patients and resistant smokers.

| | Allele* | | Genotype | | |
|---|---|---|---|---|---|
| Frequency | A | G | AA | AG | GG |
| Lung Cancer | 277 (62%) | 171 (38%) | 87 (39%) | 103 (46%) | 34 (15%) |
| n = 224 (%) | | | | | |
| Resistant | 536 (56%) | 420 (44%) | 143 (30%) | 250 (52%) | 85 (18%) |
| n = 478 (%) | | | | | |

*number of chromosomes (2n)

Genotype. AA vs AG/GG for lung cancer vs resistant, Odds ratio (OR)=1.5, 95% confidence limits 1-2.1, χ2 (Yates uncorrected)=5.5, p=0.02,

AA genotype=susceptible

Allele. A vs G for lung cancer vs resistant, Odds ratio (OR)=1.3, 95% confidence limits 1.0-1.6, χ2 (Yates uncorrected)=4.2, p=0.04,

A allele=susceptible

TABLE 13
DRD2 C/Del (rs 1799732) polymorphism allele and genotype frequencies
in the Lung cancer patients and resistant smokers.

| | Allele* | | Genotype | | |
|---|---|---|---|---|---|
| Frequency | C | Del | CC | CDel | DelDel |
| Lung Cancer | 426 (92%) | 36 (8%) | 197 (85%) | 32 (14%) | 2 (1%) |
| n = 231 (%) | | | | | |
| Resistant | 857 (89%) | 109 (11%) | 376 (78%) | 105 (22%) | 2 (0.5%) |
| n = 483 (%) | | | | | |

*number of chromosomes (2n)

Genotype. CDel/DelDel vs CC for lung cancer vs resistant, Odds ratio (OR)=0.61, 95% confidence limits 0.39-0.94, χ2 (Yates uncorrected)=5.4, p=0.02,

CDel/DelDel genotype=protective

Allele. Del vs C for lung cancer vs resistant, Odds ratio (OR)=0.66, 95% confidence limits 0.44-1.0, χ2 (Yates uncorrected)=4.2, p=0.04,

Del=protective

TABLE 14
FasL C/T (rs 763110) polymorphism allele and genotype frequencies in
the Lung cancer patients and resistant smokers.

| | Allele* | | Genotype | | |
|---|---|---|---|---|---|
| Frequency | C | T | CC | CT | TT |
| Lung Cancer | 302 (66%) | 156 (34%) | 97 (42%) | 108 (47%) | 24 (11%) |
| n = 229 (%) | | | | | |
| Resistant | 596 (61%) | 374 (39%) | 189 (39%) | 218 (45%) | 78 (16%) |
| n = 485 (%) | | | | | |

*number of chromosomes (2n)

Genotype. TT vs CC/CT for lung cancer vs resistant, Odds ratio (OR)=0.61, 95% confidence limits 0.36-1.0, χ2 (Yates uncorrected)=4.0, p=0.05,

TT genotype=protective

Fas ligand (TNF superfamily, member 6) is also known as FASLG, CD178, CD95L, TNFSF6, and APT1LG1.

TABLE 15
TLR9 C/T (rs 5743836) polymorphism allele and genotype frequencies
in the Lung cancer patients and resistant smokers.

| | Allele* | | Genotype | | |
|---|---|---|---|---|---|
| Frequency | T | C | TT | TC | CC |
| Lung Cancer | 386 (84%) | 76 (16%) | 164 (71%) | 58 (25%) | 9 (4%) |
| n = 231 (%) | | | | | |
| Resistant | 791 (85%) | 139 (15%) | 332 (71%) | 127 (27%) | 6 (1%) |
| n = 465 (%) | | | | | |

*number of chromosomes (2n)

Genotype. CC vs TC/TT for lung cancer vs resistant, Odds ratio (OR)=3.1, 95% confidence limits 1.0-9.9, χ² (Yates uncorrected)=5.0, p=0.03,

CC genotype=susceptible

TABLE 16
Summary table of protective and susceptibility polymorphisms for lung cancer.

| Gene and SNP | rs number | Genotype | Phenotype | OR | P value |
|---|---|---|---|---|---|
| Cerberus 1 (Cer 1) R19W A/G ¹ | rs10115703 | AA/AG | susceptiblility | 1.7 | 0.02 |
| XRCC4 Ser307Ser G/T ¹ | rs1056503 | GG/GT | susceptiblility | 1.3 | 0.04 |
| BRCA2 K3326X A/T ¹ | rs11571833 | AT/TT | susceptiblility | 2.5 | 0.04 |
| Integrin alpha-11 V433M A/G ¹ | rs2306022 | AA | susceptiblility | 4.3 | 0.002 |
| CAMKK1 E375G T/C ¹ | rs7214723 | TT | protective | 0.76 | 0.13 |
| P73 | rs2273953 | CC | protective | 0.46 | <0.001 |
| CYP3A43 C74 delA | | AT/TT | susceptiblility | 1.74 | 0.05 |
| BCL2 | rs2279115 | AA | protective | 0.69 | 0.05 |
| ITGB3 | rs2317676 | AG/GG | protective | 0.57 | 0.02 |
| DAT1 | rs6413429 | GT/TT | susceptibility | 1.6 | 0.05 |
| TNFR1 | rs1139417 | AA | susceptibility | 1.5 | 0.02 |
| DRD2 | rs1799732 | CDel/DelDel | protective | 0.61 | 0.02 |
| FasL | rs763110 | TT | protective | 0.61 | 0.05 |
| TLR9 | rs5743836 | CC | susceptibility | 3.1 | 0.03 |

1—included in the 5 SNP panel described below.
Odds ratios and P values derived from univariate analyses described above.

SNP scores for each subject were derived by assigning a score of +1 for the presence of susceptiblility genotypes or −1 for the presence of protective genotypes of the 5 SNPs included in the panel as identified in Table 16 above. The scores are added to derive the total SNP score for each subject. Table 17 below shows the distribution of SNP scores derived from the 5 SNP panel amongst the lung cancer patients and the resistant smoker controls.

TABLE 17
Distribution of SNP scores (5 SNP panel) in smokers with and without
lung cancer.

| | Lung cancer SNP score—5 SNP panel | | | |
|---|---|---|---|---|
| Cohort | −1 | 0 | 1 | 2 |
| Lung cancer | 33 (14%) | 119 (50%) | 75 (31%) | 12 (5%) |
| N = 239 (%) | | | | |
| Control smokers | 104 (21%) | 264 (54%) | 100 (21%) | 16 (3%) |
| N = 484 (%) | | | | |
| % with lung cancer | 33/137 | 119/383 | 75/175 | 12/28 |
| | (24%) | (31%) | (43%) | (43%) |

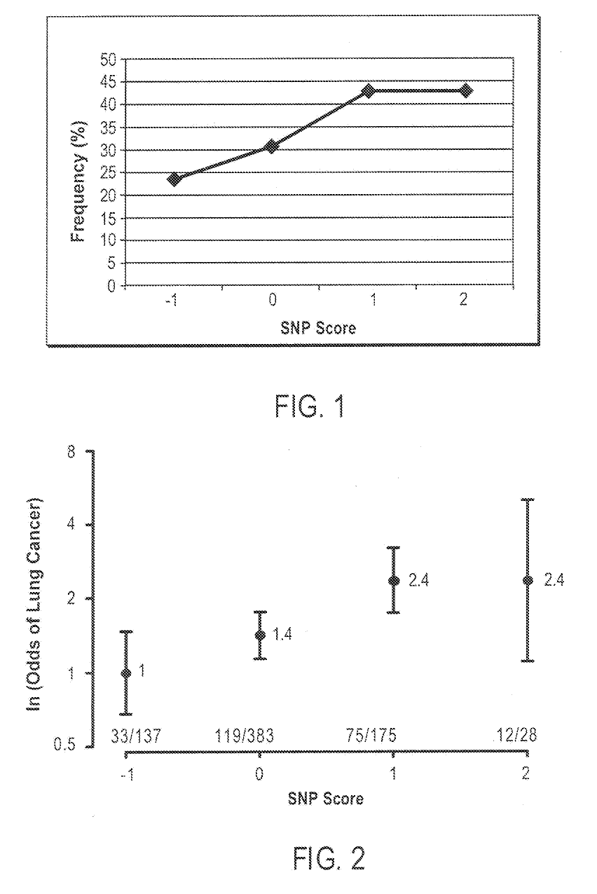

The likelihood of having lung cancer according to the lung cancer SNP score generated from the 5 SNP panel is shown graphically in FIG. 1. The log odds of having lung cancer according to the SNP score derived from the 5 SNP panel presented in Table 17 is shown in FIG. 2.

### Example 2

This example presents an analysis of distributions of SNP scores derived for lung cancer sufferors and control resistant smokers using the polymorphisms described in Table 18 below. Table 18 presents a summary of selected protective and susceptibility SNPs identified in PCT/NZ2006/000125 (published as WO2006/123955) and related applications (New Zealand Patent Application Nos. 540203/541787/543297), and herein that were included in additional panels of SNPs.

SNPs 1-11 identified in Table 18 were included in both the 11 SNP panel A and the 16 SNP panel used to generate SNP scores as discussed below. SNPs 12-16 identified in Table 18 were included in both the 5 SNP panel described in Example 1 above, and in the 16 SNP panel used to generate SNP scores as discussed below. Odd's ratios (OR) and p values are for cancer patients compared to resistant smokers with normal lung function.

TABLE 18
Summary of selected protective and susceptibility polymorphisms

| SNP# | Gene | Polymorphism | Genotype | Phenotype | OR | P value |
|---|---|---|---|---|---|---|
| 1 | Interleukin-18 | −133 G/C | CG/GG | protective | 1.5 | 0.09 |
| | (IL-18) | | CC | susceptibility | | |
| 2 | CYP2E1 | −1053 C/T (Rsa I) | TT/TC | susceptibility | 1.9 | 0.13 |
| 3 | N-acetyltransferase 2 | Arg 197 Gln A/G | GG | susceptibility | 1.5 | 0.08 |
| | (NAT2) | | | | | |
| 4 | Interleukin 1B | −511 A/G | GG | susceptibility | 1.6 | 0.04 |
| | (IL-1B) | | | | | |
| 5 | Anti chymotrypsin | Ala 15 Thr | GG | susceptibility | 1.7 | 0.06 |
| | (ACT) | | | | | |
| 6 | α1-antitrypsin | S allele ¹ | AT/TT | susceptibility | | |
| 7 | Interleukin-8 (IL-8) | −251 A/T | AA | protective | 4.1 | 0.002 |
| 8 | XPD | Lys -751 Gln G/T | GG | protective | 1.7 | 0.18 |
| 9 | Superoxide | Arg 312 Gln | CG/GG | protective | 3.38 | 0.03 |
| | dismutase 3 (SOD3) | (+760 G/C) | | | | |
| 10 | REV1 | Phe 257 Ser C/T | CC | protective | 0.73 | 0.20 |
| 11 | α1-antitrypsin | Z allele ¹ | AG | protective | | |
| 12 | Cerberus 1 (Cer 1) | Rs 19W A/G ²) | AA/AG | susceptiblility | 1.7 | 0.02 |
| | | (rs 10115703) | | | | |
| 13 | XRCC4 | Ser307Ser G/T ² | GG/GT | susceptiblility | 1.3 | 0.04 |
| | | (rs1056503) | | | | |
| 14 | BRCA2 | K3326X A/T ² | AT/TT | susceptiblility | 2.5 | 0.04 |
| | | (rs 11571833) | | | | |
| 15 | Integrin alpha-11 | V433M A/G ² | AA | susceptiblility | 4.3 | 0.002 |
| | | (rs 2306022) | | | | |
| 16 | CAMKK1 | E375G T/C ² | TT | protective | 0.76 | 0.13 |
| | | (rs7214723) | | | | |

^{1—}discussed in PCT International application PCT/NZ2006/000125.
^{2—}included in both the 5 SNP panel (described in Example 1) and the 16 SNP panel.

Table 19 below presents the distribution of SNP scores derived from the 11 SNP panel A consisting of SNPs numbers 1 to 11 from Table 18 in the lung cancer patients and the resistant smoker controls.

TABLE 19
Distribution of the lung cancer SNP score

| | Cohort | | | | | | | | | | |
|---|---|---|---|---|---|---|---|---|---|---|---|
| | lung cancer SNP score—11 SNP panel A | | | | | | | | | | |
| | 0 | 1 | 2 | 3 | 4 | 5 | 6 | 7 | 8 | 9 | 10+ |
| Lung | 2 | 5 | 9 | 12 | 13 | 21 | 47 | 44 | 37 | 24 | 25 |
| cancer | (1%) | (2%) | (4%) | (5%) | (5%) | (9%) | (20%) | (18%) | (16%) | (10%) | (10%) |
| N = 239 | | | | | | | | | | | |
| Smoking | 23 | 45 | 74 | 69 | 48 | 51 | 68 | 58 | 31 | 14 | 3 |
| controls | (5%) | (9%) | (15%) | (14%) | (10%) | (11%) | (14%) | (12%) | (6%) | (3%) | (1%) |
| N = 484 | | | | | | | | | | | |
| % with | 6/80 | 7/68 | 8/73 | 15/72 | 26/79 | 37/107 | 37/82 | 44/79 | 29/44 | 16/22 | 14/17 |
| lung | (8%) | (10%) | (11%) | (21%) | (33%) | (37%) | (45%) | (56%) | (66%) | (73%) | (82%) |
| cancer | | | | | | | | | | | |

The shaded SNP scores (0, 1, and 2) can be viewed as low to average risk of lung cancer. At this threshold (cut-off), 7% of lung cancer cases were present, while 29% of the control smokers were present. On the graph plotting lung cancer frequency versus SNP score (FIG. 3), this equates to an approximately 10% risk of lung cancer. This is the average across all smokers. The likelihood of having lung cancer according to the SNP score derived from the 11 SNP panel A is shown in FIG. 3.

The distribution of SNP scores among lung cancer patients and resistant smoker controls were further analysed as follows. FIG. 4 depicts a receiver—operator curve analysis with sensitivity and sensitivity for the lung cancer 11 SNP panel A. This was developed according to the model:

(IL18_133_S+CYP2E1_Rsal_S+NAT2_197_S+IL1B_511_S+ACT_15_S+s_allele_S+IL8_251_S+z_allele_s)
−
(XPD_75 l_P+SOD3_213_P+REV1_257_P)
if age >60 then add 4
if FHx lung Ca then add 3

| Area under the ROC curve | Results |
|---|---|
| Area | 0.7483 |
| Std. Error | 0.01907 |
| 95% confidence interval | 0.7109 to 0.7856 |
| P value | <0.0001 |

| | | | | | Likelihood |
|---|---|---|---|---|---|
| Cutoff | Sensitivity | 95% CI | Specificity | 95% CI | ratio |
| >−0.5000 | 0.9958 | 0.9769 to 0.9999 | 0.004132 | 0.0005008 to 0.01485 | 1.00 |
| >0.5000 | 0.9916 | 0.9701 to 0.9990 | 0.04752 | 0.03036 to 0.07045 | 1.04 |
| >1.500 | 0.9707 | 0.9406 to 0.9881 | 0.1405 | 0.1108 to 0.1747 | 1.13 |
| >2.500 | 0.9331 | 0.8936 to 0.9613 | 0.2934 | 0.2532 to 0.3362 | 1.32 |
| >3.500 | 0.8828 | 0.8351 to 0.9207 | 0.4360 | 0.3913 to 0.4814 | 1.57 |
| >4.500 | 0.8285 | 0.7746 to 0.8740 | 0.5351 | 0.4896 to 0.5803 | 1.78 |
| >5.500 | 0.7406 | 0.6801 to 0.7950 | 0.6405 | 0.5960 to 0.6833 | 2.06 |
| >6.500 | 0.5439 | 0.4785 to 0.6083 | 0.7810 | 0.7415 to 0.8171 | 2.48 |
| >7.500 | 0.3598 | 0.2990 to 0.4242 | 0.9008 | 0.8707 to 0.9260 | 3.63 |
| >8.500 | 0.2050 | 0.1557 to 0.2618 | 0.9649 | 0.9444 to 0.9794 | 5.84 |
| >9.500 | 0.1046 | 0.06884 to 0.1505 | 0.9938 | 0.9820 to 0.9987 | 16.88 |
| >10.50 | 0.03766 | 0.01736 to 0.07028 | 0.9979 | 0.9885 to 0.9999 | 18.23 |
| >11.50 | 0.004184 | 0.0001059 to 0.02309 | 1.000 | 0.9924 to 1.000 | |

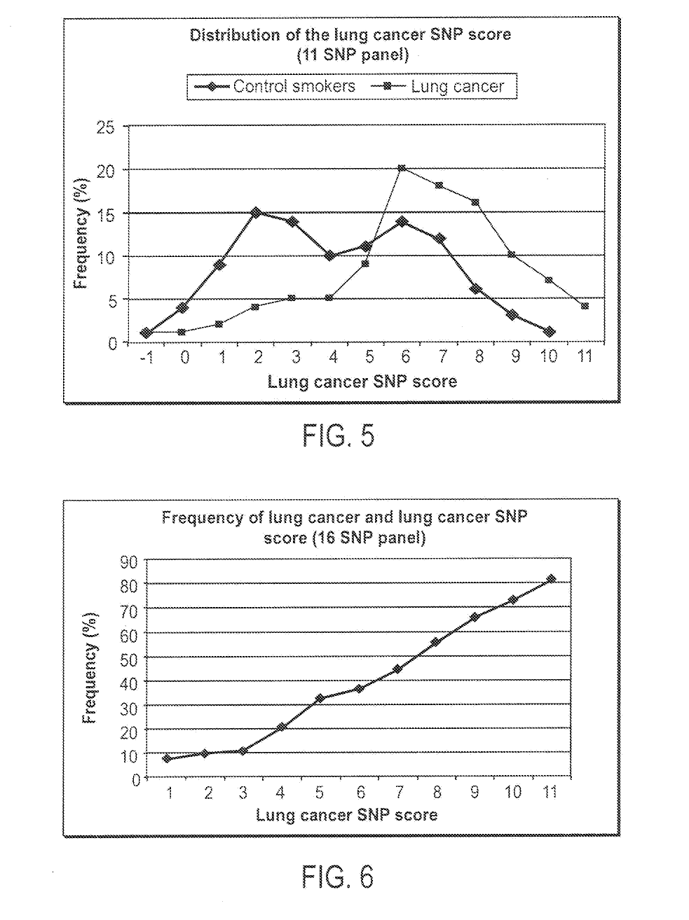

FIG. 5 herein presents a graph showing the distribution of SNP score derived from the 11 SNP panel A among lung cancer sufferers and among resistant smoker controls.

TABLE 20
Distribution of the lung cancer SNP score derived from the 16 SNP panel

| | 16 SNP lung cancer SNP score | | | | | | | | | | |
|---|---|---|---|---|---|---|---|---|---|---|---|
| | ≦1 | 2 | 3 | 4 | 5 | 6 | 7 | 8 | 9 | 10 | 11+ |
| Lung | 6 | 7 | 8 | 15 | 26 | 37 | 37 | 44 | 29 | 16 | 14 |
| cancer | (2%) | (3%) | (3%) | (6%) | (11%) | (15%) | (15%) | (18%) | (12%) | (7%) | (6%) |
| N = 239 | | | | | | | | | | | |
| Smoking | 74 | 61 | 65 | 57 | 53 | 70 | 45 | 35 | 15 | 6 | 3 |
| controls | (15%) | (13%) | (13%) | (12%) | (11%) | (15%) | (9%) | (7%) | (3%) | (1%) | (1%) |
| N = 484 | | | | | | | | | | | |
| % with | 6/80 | 7/68 | 8/73 | 15/72 | 26/79 | 37/107 | 37/82 | 44/79 | 29/44 | 16/22 | 14/17 |
| lung | (8%) | (10%) | (11%) | (21%) | (33%) | (37%) | (45%) | (56%) | (66%) | (73%) | (82%) |
| cancer | | | | | | | | | | | |

The shaded SNP scores (≦1, 2, and 3) can be viewed as low to average risk of lung cancer. At this cut-off, 8% of lung cancer cases were present, while 41% of control smokers were present. On the graph plotting lung cancer frequency and SNP score (FIG. 6), this equates to about a 10% risk of lung cancer, the average across all smokers. The likelihood of having lung cancer according to the SNP score derived from the 16 SNP panel is shown in FIG. 6.

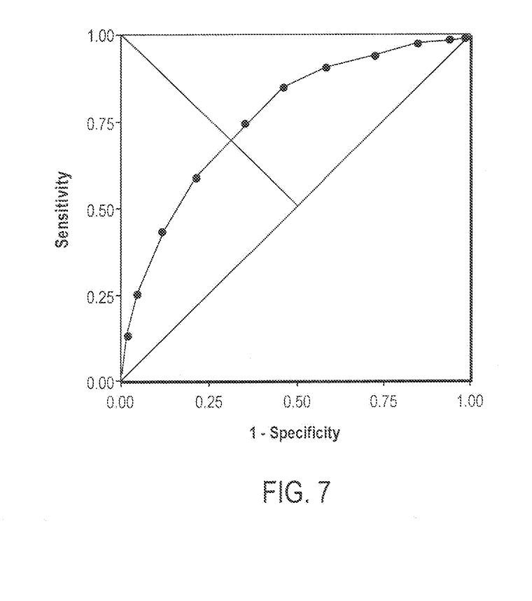

The distribution of SNP scores among lung cancer patients and resistant smoker controls were further analysed as follows. FIG. 7 depicts a receiver—operator curve analysis with sensitivity and sensitivity for the lung cancer 16 SNP panel. This was developed according to the model:

(IL18_133_S+CYP2E1_Rsal_S+NAT2_197_S+IL1B_511_S+ACT_15_S+s_allele_S+IL8_251_S+z_allele_s)
−(XPD_751_P+SOD3_213P+REV1_257_P)
+
(ITGA11_s+Cerl_s+BRAC2_s+XRCC4_307_s)

### −CAMKK1_p

if age >60 then add 4
if FHx lung Ca then add 3

| Area under the ROC curve | Results |
|---|---|
| Area | 0.7621 |
| Std. Error | 0.01855 |
| 95% confidence interval | 0.7257 to 0.7985 |
| P value | <0.0001 |

| | | | | | Likelihood |
|---|---|---|---|---|---|
| Cutoff | Sensitivity | 95% CI | Specificity | 95% CI | ratio |
| >−0.5000 | 0.9958 | 0.9769 to 0.9999 | 0.01240 | 0.004563 to 0.02679 | 1.01 |
| >0.5000 | 0.9874 | 0.9638 to 0.9974 | 0.05992 | 0.04049 to 0.08492 | 1.05 |
| >1.500 | 0.9749 | 0.9462 to 0.9907 | 0.1529 | 0.1220 to 0.1881 | 1.15 |
| >2.500 | 0.9456 | 0.9088 to 0.9707 | 0.2789 | 0.2394 to 0.3212 | 1.31 |
| >3.500 | 0.9121 | 0.8688 to 0.9448 | 0.4132 | 0.3690 to 0.4585 | 1.55 |
| >4.500 | 0.8494 | 0.7976 to 0.8922 | 0.5310 | 0.4854 to 0.5762 | 1.81 |
| >5.500 | 0.7406 | 0.6801 to 0.7950 | 0.6405 | 0.5960 to 0.6833 | 2.06 |
| >6.500 | 0.5858 | 0.5205 to 0.6489 | 0.7851 | 0.7458 to 0.8209 | 2.73 |
| >7.500 | 0.4310 | 0.3673 to 0.4964 | 0.8781 | 0.8456 to 0.9059 | 3.54 |
| >8.500 | 0.2469 | 0.1935 to 0.3066 | 0.9504 | 0.9271 to 0.9680 | 4.98 |
| >9.500 | 0.1255 | 0.08632 to 0.1743 | 0.9814 | 0.9650 to 0.9915 | 6.75 |
| >10.50 | 0.05858 | 0.03239 to 0.09633 | 0.9938 | 0.9820 to 0.9987 | 9.45 |
| >11.50 | 0.02092 | 0.006827 to 0.04814 | 1.000 | 0.9924 to 1.000 | |

FIG. 8 herein presents a graph showing the distribution of SNP score derived from the 16 SNP panel among lung cancer sufferers and among resistant smoker controls.

### Example 3

This example presents a multivariate analysis using a 9 SNP panel comprising the polymorphisms described in Table 21 below. Table 21 summarises the univariate analysis showing protective and susceptibility SNPs associated with lung cancer as set out in Tables 7-15. Odd's ratios (OR) and p values are for cancer patients compared to resistant smokers with normal lung function.

TABLE 21
Summary of selected polymorphisms—9 SNP panel

| Gene and SNP | rs number | Genotype | Phenotype | OR | P value |
|---|---|---|---|---|---|
| P73 | rs2273953 | CC | protective | 0.46 | <0.001 |
| CYP3A43 | | AT/TT | susceptiblility | 1.74 | 0.05 |
| C74 delA | | | | | |
| BCL2 | rs2279115 | AA | protective | 0.69 | 0.05 |
| ITGB3 | rs2317676 | AG/GG | protective | 0.57 | 0.02 |
| DAT1 | rs6413429 | GT/TT | susceptibility | 1.6 | 0.05 |
| TNFR1 | rs1139417 | AA | susceptibility | 1.5 | 0.02 |
| DRD2 | rs1799732 | CDel/ | protective | 0.61 | 0.02 |
| | | DelDel | | | |
| FasL | rs763110 | TT | protective | 0.61 | 0.05 |
| TLR9 | rs5743836 | CC | susceptibility | 3.1 | 0.03 |

As described above in respect of the 5, 11, and 16 SNP panels, a SNP score was determined for each subject from the univariate data for this 9 SNP panel. The presence of the susceptibility SNP genotype was scored +1, and the presence of the protective SNP genotype was scored −1.

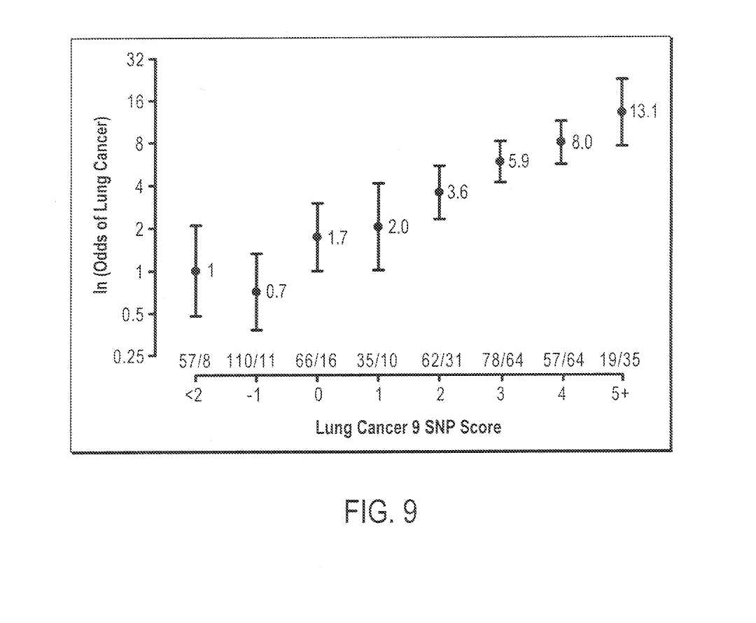

As shown in FIG. 9, a linear relationship was observed when the SNP score for lung cancer patients and healthy smoking controls were analysed together and plotted according to the odds of having lung cancer, where those with the highest scores have the greatest risk. In this analysis (floating absolute odds ratio), the lowest SNP score group is referenced as 1. Those with the highest score (5 or more) have an Odds of 13—they are at 13 fold greater likelihood (or risk) of being diagnosed with lung cancer.

For each subject, a composite score that defines a likelihood of being diagnosed with lung cancer was derived. The SNP score from the 9 SNP panel was combined with scores according to age (+4 for age over 60 yo) and family history (+3 for having a first degree relative with lung cancer) for each subject. This algorithm generated a composite score for each smoker based on genotype, age and family history of lung cancer. Table 22 below shows the results of this multivariate analysis using these 9 SNPs, age and family history.

TABLE 22
Multivariate analysis

| Analysis of Maximum Likelihood Estimates | | | | | | | | |
|---|---|---|---|---|---|---|---|---|
| | | | | Wald | | | 95% Wald | |
| | | | Standard | Chi- | Pr > | | Confidence | |
| Parameter | DF | Estimate | Error | Square | ChiSq | OR | Limits | |
| Intercept | 1 | 4.1002 | 0.8241 | 24.7553 | <.0001 | | | |
| P73_p | 1 | 0.7646 | 0.1995 | 14.6902 | 0.0001 | 2.148 | 1.453 | 3.176 |
| DRD2_p | 1 | 0.6471 | 0.2639 | 6.0107 | 0.0142 | 1.910 | 1.139 | 3.204 |
| BCL2_p | 1 | 3.3845 | 0.2310 | 2.7711 | 0.0960 | 1.469 | 3.934 | 2.310 |
| FasL_p | 1 | 0.8187 | 0.2991 | 7.4906 | 0.0062 | 2.267 | 1.262 | 4.075 |
| ITGB3_p | 1 | 0.7764 | 0.2985 | 6.7636 | 0.0093 | 2.174 | 1.211 | 3.902 |
| TNFR1_s | 1 | −0.1094 | 0.2180 | 0.2517 | 0.6159 | 0.896 | 0.585 | 1.374 |
| CYP3A43_s | 1 | −0.7760 | 0.3741 | 4.3036 | 0.0380 | 0.460 | 0.221 | 0.958 |
| DAT1_s | 1 | −0.4273 | 0.2918 | 2.1431 | 0.1432 | 0.652 | 0.368 | 1.156 |
| TLR9_s | 1 | −0.6429 | 0.6268 | 1.0520 | 0.3050 | 0.526 | 0.154 | 1.796 |
| Age | 1 | −0.0796 | 0.0104 | 58.3869 | <.0001 | 0.923 | 0.905 | 0.943 |
| FHxLCancer | 1 | 0.3105 | 0.2582 | 1.4452 | 0.2293 | 1.364 | 0.822 | 2.263 |
| | | c | 0.770 | | | | | |

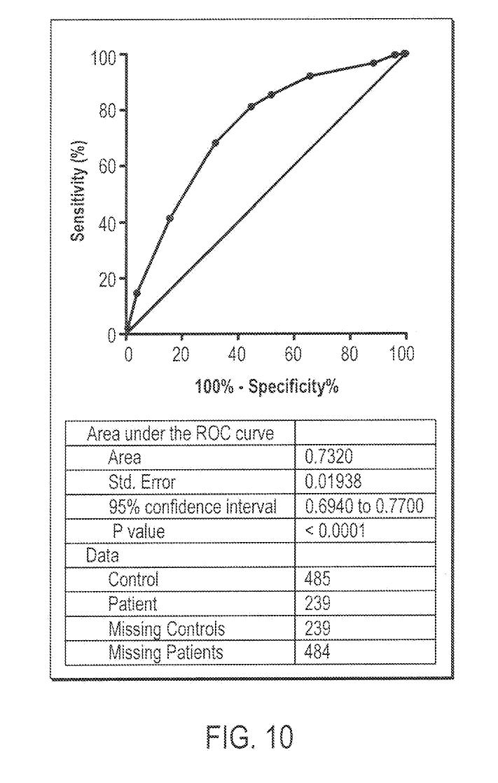

FIG. 10 shows the receiver-operator curve analysis for this composite lung cancer SNP score. The receiver operator curve analysis shows the area under the ROC curve is 0.73 for these 9 SNPs. This indicates an acceptable level of discrimination.

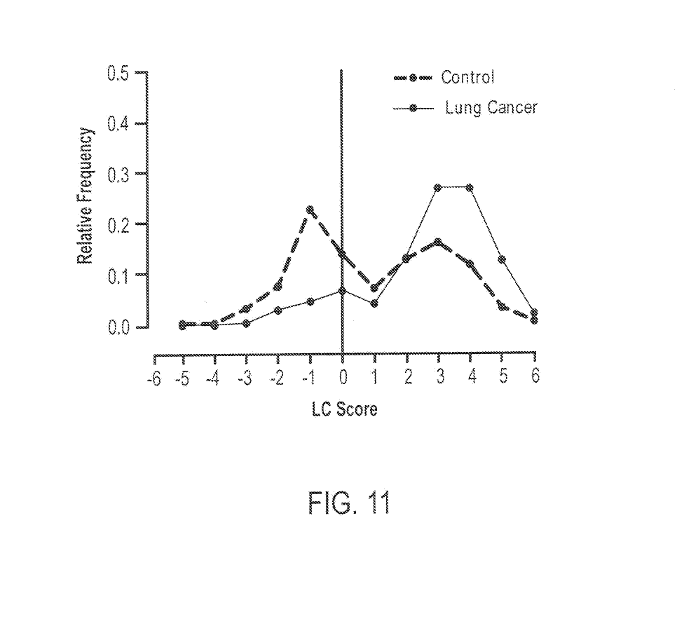

When the frequency distribution for the 9 SNP panel SNP score is compared between lung cancer cases and controls (FIG. 11), separation of the lung cancer SNP score between cases and controls is observed. This reflects the ability of the SNP score to discriminate between high and low risk smokers. This data shows that SNPs on their own derive modest levels of risk (small Odds ratios). These SNPs can be analysed in combination to derive a risk score with clinical utility in discriminating smokers at high and low risk of lung cancer based on their genotype, and such analyses can include non-genetic factors such as age and family history.

### Example 4

This example presents a multivariate analysis using an 11 SNP panel (11 SNP panel B) comprising the polymorphisms described in Table 23 below. Table 23 summarises the univariate analysis showing protective and susceptibility SNPs associated with lung cancer as set out herein. Odd's ratios (OR) and p values are for cancer patients compared to resistant smokers with normal lung function. Stepwise regression analysis was also performed, and chi squared values are presented for each polymorphism.

TABLE 23
Summary of Selected Polymorphisms—11 SNP Panel B

| | | Lung | Smoking | Call | Univariate | | Stepwise | | |
|---|---|---|---|---|---|---|---|---|---|
| SNP (rs#) | Genotype | cancer | controls | rate | OR | P value | regression χ2 | P value | Phenotype |
| Interleukin-18 | CC | 237 (54%) | 208 (45%) | 96% | 1.4 | 0.009 | 10.4 | 0.001 | susceptibility |
| (−133 G/C) | CG/GG | 201 (46%) | 250 (55%) | | (1.1-1.9) | | | | |
| Interleukin-8 | TT | 129 (31%) | 109 (23%) | 96% | 1.5 | 0.005 | 6.5 | 0.01 | susceptibility |
| (−251 A/T) | AT/AA | 284 (69%) | 367 (77%) | | (1.1-2.1) | | | | |
| ITGA11 | AA | 14 (3%) | 6 (1%) | 98% | | 2.6 | 0.04 | | susceptibility |
| (rs2306022) | GA/GG | 422 (97%) | 470 (99%) | | (0.9-7.6) | | | | |
| N-acetylcysteine | GG | 239 (56%) | 222 (47%) | 97% | 1.4 | 0.006 | 5.8 | 0.02 | susceptibility |
| transferase 2 | AA/AG | 189 (44%) | 253 (53%) | | (1.1-1.9) | | | | |
| (rs 1799930) | | | | | | | | | |
| α1-antichymotrypsin | GG | 123 (28%) | 96 (20%) | 98% | 1.6 | 0.004 | 7.1 | 0.008 | susceptibility |
| (−15 A/G) | AG/AA | 312 (72%) | 383 (80%) | | (1.2-2.2) | | | | |
| DAT1 | GT/TT | 64 (15%) | 50 (10%) | 98% | 1.5 | 0.04 | 4.2 | 0.04 | susceptibility |
| (rs6413429) | GG | 367 (85%) | 431 (90%) | | (1.0-2.3) | | | | |
| P73 | CC | 219 (52%) | 292 (62%) | 96% | 0.65 | 0.001 | 11.8 | 0.0006 | protective |
| (rs 2273953) | TC/TT | 206 (48%) | 178 (38%) | | (0.49-0.85) | | | | |
| SOD3 | GG/GC | 4 (1%) | 15 (3%) | 96% | 0.28 | 0.02 | 7.7 | 0.005 | protective |
| (rs1799895) | CC | 425 (99%) | 451 (97%) | | (0.10-0.90) | | | | |
| ITGB3 | GG/GA | 44 (10%) | 77 (16%) | 98% | 0.59 | 0.008 | 6.6 | 0.01 | protective |
| (rs2317676) | AA | 391 (90%) | 403 (84%) | | (0.39-0.89) | | | | |
| DRD2 | CDel/Del.Del | 70 (16%) | 107 (22%) | 98% | 0.68 | 0.02 | 7.3 | 0.007 | protective |
| (rs 1799732) | CC | 359 (84%) | 372 (78%) | | (0.48-0.96) | | | | |
| BCL2 | AA | 103 (24%) | 145 (31%) | 97% | 0.71 | 0.03 | 4.2 | 0.04 | protective |
| (rs 2279115) | AC/CC | 328 (76%) | 330 69%) | | (0.53-0.97) | | | | |

As described above, a SNP score was determined for each subject from the univeriate data for the 11 SNP panel B. The presence of the susceptibility SNP genotype was scored +1, and the presence of the protective SNP genotype was scored −1.

For each subject, a score that defines a likelihood of being diagnosed with lung cancer was derived. Table 23 above shows the results of this multivariate analysis using these 11 SNPS and indicates these SNPs can be analysed in combination to derive a risk score with clinical utility in discriminating smokers at high and low risk of lung cancer based on their genotype.

### Discussion

The above results show that several polymorphisms were associated with either increased or decreased risk of developing lung cancer. The associations of individual polymorphisms on their own, while of discriminatory value, are unlikely to offer an acceptable prediction of disease. However, in combination these polymorphisms distinguish susceptible subjects from those who are resistant (for example, between the smokers who develop lung cancer and those with the least risk with comparable smoking exposure). The polymorphisms represent exonic polymorphisms known to alter amino-acid sequence (and likely expression and/or function) in a number of genes involved in processes known to underlie lung remodelling and lung cancer, and in one case a silent mutation having no effect on amino acid composition. The polymorphisms identified here are found in genes encoding proteins central to these processes which include inflammation, matrix remodelling, oxidant stress, DNA repair, cell replication and apoptosis.

In the comparison of smokers with lung cancer and matched smokers with near normal lung function (lowest risk for lung cancer despite smoking), several polymorphisms were identified as being found in significantly greater or lesser frequency than in the comparator groups (sometimes including the blood donor cohort). Due to the small cohort of lung cancer patients, polymorphisms where there are only trends towards differences (P=0.06-0.25) were included in the analyses, although in the combined analyses only those polymorphisms with the most significant differences were utilised.
In the analysis of the R19W A/G polymorphism of the Cerberus 1 gene, the AA and AG genotypes were found to be significantly greater in the lung cancer cohort compared to the resistant smoker cohort (OR=1.7, P=0.02), consistent with each having a susceptibility role (see Table 2). The A allele was found to be significantly greater in the lung cancer cohort compared to the resistant smoker cohort (OR=1.5, P=0.05), consistent with a susceptibility role. In contrast, the GG genotype was found to be greater in the resistant smoker control cohort compared to the lung cancer cohort, consistent with a protective role (see Table 2).
In the analysis of the Ser307Ser G/T polymorphism in the XRCC4 gene, the GG and GT genotypes were found to be greater in the lung cancer cohort compared to the resistant smoker cohort (OR=1.3, P=0.12) consistent with each having a susceptibility role. The G allele was found to be significantly greater in the lung cancer cohort compared to the resistant smoker controls (OR=1.4, P=0.04), consistent with a suscepbility role (see Table 3). In contrast, the TT genotype was found to be greater in the resistant smoker control compared to the lung cancer cohort, consistent with a protective role.
In the analysis of the K3326X A/T polymorphism in the ERCA2 gene, the A/T and TT genotypes were found to be significantly greater in the lung cancer cohort compared to the resistant smoker controls (OR=2.5, P=0.04), consistent with a suscepbility role. The T allele was found to be significantly greater in the lung cancer cohort compared to the resistant smoker controls (OR=2.7, P=0.02), see Table 4. In contrast the AA genotype was found to be greater in the resistant smoker controls compared to the lung cancer cohort, consistant with a protective role.
In the analysis of the V433M A/G polymorphism, in the Integrin alpha-11 gene, the AA genotype was found to be significantly greater in the lung cancer cohort compared to the resistant smoker controls (OR=4.3, P=0.002) consistent with a susceptibility role (see Table 5). The A allele was found to be significantly greater in the lung cancer cohort compared to the resistant smoker controls (OR=1.4, P=0.04), consistent with a susceptibility role (see Table 5).
In the analysis of the E375G T/C polymorphism in the Calcium/calmodulin-dependent protein kinase kinase 1 gene, the TT genotype was found to be greater in the resistant smoker controls compared to the lung cancer cohort (OR=0.76, P=0.13), consistent with a protective role (see Table 6). The T allele is found to be greater in resistant smoker controls compared to the lung cancer cohort (OR=0.84, P=0.14), consistent with a protective role (see Table 6).
In the analysis of the −81 C/T (rs 2273953) polymorphism in the 5′ UTR of the gene encoding Tumor protein P73, the CC genotype was found to be significantly greater in the resistant smoker cohort compared to the lung cancer cohort (OR=0.46, P<0.001) consistent with a protective role. The C allele was also found to be significantly greater in the resistant smoker controls compared to the lung cancer cohort (OR=0.62, P<0.001), consistent with a protective role (see Table 7). In contrast, the CT and TT genotypes were found to be greater in the the lung cancer cohort compared to resistant smoker controls, consistent with a susceptibility role.
In the analysis of the A/T c74delA polymorphism in the gene encoding cytochrome P450 polypeptide CYP3A43, the AT and TT genotypes were found to be significantly greater in the lung cancer cohort compared to the resistant smoker cohort (OR=1.74, P=0.05), consistent with each having a susceptibility role (see Table 8). The T allele was found to be significantly greater in the lung cancer cohort compared to the resistant smoker cohort (OR=1.8, P=0.03), also consistent with a susceptibility role.
In the analysis of the A/C (rs2279115) polymorphism in the gene encoding B-cell CLL/lymphoma 2, the AA genotype was found to be significantly greater in the resistant smoker cohort compared to the lung cancer cohort (OR=0.69, P=0.05) consistent with a protective role. The A allele was also found to be significantly greater in the resistant smoker controls compared to the lung cancer cohort (OR=0.78, P=0.02), consistent with a protective role (see Table 9).
In the analysis of the A/G at +3100 polymorphism in the 3′UTR (rs2317676) of the gene encoding Integrin beta 3, the AG and GG genotypes were found to be significantly greater in the resistant smoker cohort compared to the lung cancer cohort (OR=0.57, P=0.02) consistent with a protective role. The G allele was also found to be significantly greater in the resistant smoker controls compared to the lung cancer cohort (OR=0.54, P=0.01), consistent with a protective role (see Table 10).
In the analysis of the −3714 G/T (rs6413429) polymorphism in the gene encoding Dopamine transporter 1, the TT and GT genotypes were found to be significantly greater in the lung cancer cohort compared to the resistant smoker cohort (OR=1.6, P=0.05), consistent with each having a susceptibility role (see Table 11).
In the analysis of the A/G (rs1139417) polymorphism in the gene encoding Tumor necrosis factor receptor 1, the AA genotype was found to be significantly greater in the lung cancer cohort compared to the resistant smoker cohort (OR=1.5, P=0.02), consistent with a susceptibility role (see Table 12). The A allele was found to be significantly greater in the lung cancer cohort compared to the resistant smoker cohort (OR=1.3, P=0.04), also consistent with a susceptibility role.
In the analysis of the C/Del (rs1799732) polymorphism in the gene encoding Dopamine receptor D2, the CDel and DelDel genotypes were found to be significantly greater in the resistant smoker cohort compared to the lung cancer cohort (OR=0.61, P=0.02) consistent with each having a protective role. The Del allele was also found to be significantly greater in the resistant smoker controls compared to the lung cancer cohort (OR=0.66, P=0.04), consistent with a protective role (see Table 13).
In the analysis of the C/T (rs763110) polymorphism in the gene encoding Fas ligand, the TT genotype was found to be significantly greater in the resistant smoker cohort compared to the lung cancer cohort (OR=0.61, P=0.05) consistent with a protective role (see Table 14).
In the analysis of the C/T (rs5743836) polymorphism in the gene encoding Toll-like receptor 9, the CC genotype was found to be significantly greater in the lung cancer cohort compared to the resistant smoker cohort (OR=3.1, P=0.02), consistent with a susceptibility role (see Table 15).

It is accepted that the disposition to lung cancer is the result of the combined effects of the individual's genetic makeup and other factors, including their lifetime exposure to various aero-pollutants including tobacco smoke. Similarly it is accepted that lung cancer encompasses several obstructive lung diseases and characterised by impaired expiratory flow rates (eg FEV1). The data herein suggest that several genes can contribute to the development of lung cancer. A number of genetic mutations working in combination either promoting or protecting the lungs from damage are likely to be involved in elevated resistance or susceptibility to lung cancer.

From the analyses of the individual polymorphisms, 6 protective genotype and 8 susceptibility genotypes were identified and analysed for their frequencies in the smoker cohort consisting of resistant smokers and those with lung cancer. A SNP score was determined for each subject by assigning a score of +1 for the presence of a suscepbility genotype and −1 for the presence of a protective genotype. These scores were added to derive a SNP score for each subject.

When the frequency of resistant smokers and smokers with lung cancer were compared according to the SNP score derived from a 5 SNP panel consisting of the SNPs identified in Table 16 herein, the chances of having lung cancer increased from 24%-31% to 43% in smokers with a SNP score of −1, 0, or 1+, respectively. When the frequencies of resistant smokers and smokers with lung cancer were compared according to a SNP score derived from an 11 SNP panel (11 SNP panel A), it was found that the chances of having lung cancer increased from 8% to 82% in smokers with a SNP score of 0 compared to those with a SNP score of 10+.

A minor increase in the linearity of the relationship between SNP score and frequency of lung cancer was observed when the SNP score was derived from a 16 SNP panel consisting of the SNPs identified in Table 18 herein. Again, the chances of having lung cancer increased from 8%, to 82% in smokers with a SNP score of less than or equal to 1 compared to those with a SNP score of 11+. The slight increase in linearity can be seen in a comparison of FIG. 3 (11 SNP panel B) and FIG. 4 (16 SNP panel).

When the frequency of resistant smokers and smokers with lung cancer were compared according to the SNP score derived from a 9 SNP panel consisting of the SNPs identified in Table 21 herein, the chances of having lung cancer was increased 13-fold in smokers with a SNP score of 5+ compared to those with a SNP score of 1.

These findings indicate that the methods of the present invention may be predictive of lung cancer in an individual well before symptoms present.

Importantly, a substantial difference is seen in the distribution of lung cancer patients and control smokers relative to total SNP score when the SNP score is derived from the 16 SNP panel rather than from the 11 SNP panel B (see FIG. 8 compared to FIG. 5). In this analysis, the addition of the 5 SNPs discussed herein to the 11 SNP panel B results in only a small change to the linear relationship between lung cancer SNP score and frequency of lung cancer for the 11 SNP panel B compared to the 16 SNP panel (see FIGS. 3 and 6, respectively), and results in only a small difference to the receiver-operator curve analysis with sensitivity and specificity (see FIGS. 4 and 7, respectively). However, this addition results in a substantial difference to the utility of the SNP score, and identifies a larger subgroup of control smokers who are “low risk” defined by a cut off over the linear scale of SNP score (see FIG. 8 compared to FIG. 5). A similarly useful discrimination between lung cancer sufferors and resistant controls was observed when a distribution of SNP scores calculated using the 9 SNP panel was derived—see FIG. 11. This has important implications in rationing or prioritising medical interventions.

These findings indicate that the methods of the present invention may be used to identify subsets of nominally at risk individuals (and particularly smokers) who are at low to average risk of lung cancer, and are thus not suitable for an intervention.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. In another example, a given susceptibility genotype is associated with increased expression of a gene relative to that observed with the protective genotype. A suitable therapy in subjects known to possess the susceptibility genotype is the administration of an agent capable of reducing expression of the gene, for example using antisense or RNAi methods. An alternative suitable therapy can be the administration to such a subject of an inhibitor of the gene product. In still another example, a susceptibility genotype present in the promoter of a gene is associated with increased binding of a repressor protein and decreased transcription of the gene. A suitable therapy is the administration of an agent capable of decreasing the level of repressor and/or preventing binding of the repressor, thereby alleviating its downregulatory effect on transcription. An alternative therapy can include gene therapy, for example the introduction of at least one additional copy of the gene having a reduced affinity for repressor binding (for example, a gene copy having a protective genotype).

Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method comprises the detection of the presence or absence of a susceptibility polymorphism which when present either upregulates or downregulates expression of the gene such that a state of such excess or deficit is the outcome, with those subjects in which the polymorphism is present being likely responders to treatment.

### Example 5

This example describes the analysis of the relationship between SNP score and risk of the four most common types of lung cancer.

The lung cancer cohort described in Example 1 above is typical of that seen in other reported lung cancer studies. In particular, the distribution of the four leading histological types of primary lung cancer is consistent with larger studies. Here, 45% of subjects had adenocarcinoma, 23% of subjects had squamous cell lung cancer, 16% of subjects had small cell lung cancer, and 13% of subjects had non-small cell lung cancer.

Reporters of epidemiological studies have suggested that smoking plays a greater role in small cell and squamous cell lung cancer and less in adenocarcinoma. The basis of this suggestion is not certain. The role of genetic factors in each histological type of lung cancer is unknown.

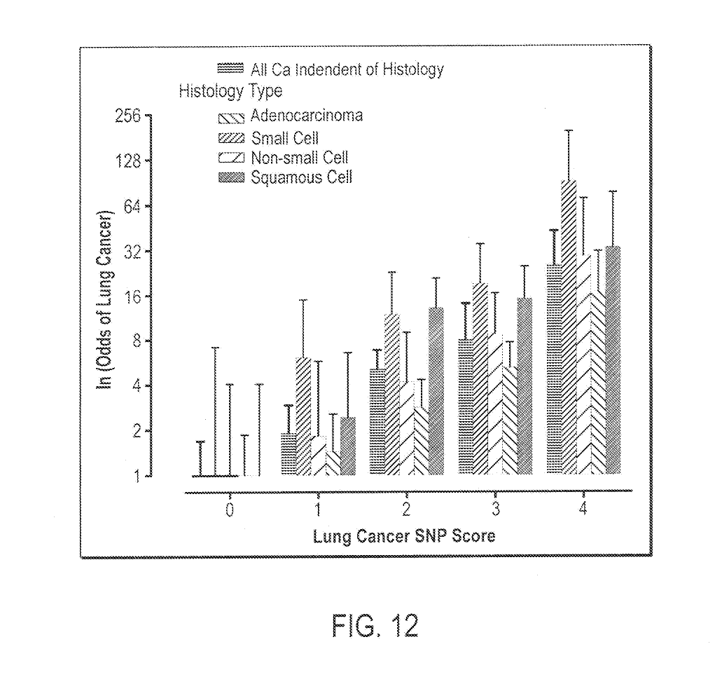

When the relationship between SNP score (determined as described above) and risk of lung cancer was examined according to histological type, the risk (Odds ratio) is higher for those with small-cell lung cancer and squamous cell lung cancer while least for those with adenocarcinoma (see FIG. 12).

Without wishing to be bound by any theory, this suggests that the genetic effect measured by the SNP score may interact with smoking to confer risk of lung cancer. It also suggests, again without wishing to be bound by any theory, that the SNP score effect, although present, is least for lung cancer of the adenocarcinoma type (typically seen in light smokers or non-smokers). Collectively this example shows that the SNP score has utility in identifying those at risk of all types of lung cancer, and that an analysis of SNP score may be useful in determining not only whether or not an intervention in respect of a subject is warranted or desirable, but also the type of intervention. For example, on the basis of their SNP score, a subject may be considered suitable for more frequent screening (e.g., for rapidly-growing or aggressive lung cancer types).

### Example 6

This example presents the identification and analysis of a 19 SNP panel (11 susceptibility SNPs) and 8 protective SNPs as shown in Table 24 below useful for the methods of the present invention.

Statistical Analysis

Patient characteristics in the lung cancer sufferers and controls were compared by unpaired t-tests for continuous variables and chi-square test or Fisher's exact test for discrete variables. Genotype and allele frequencies were checked for Hardy Weinberg Equilibrium and population admixture by the Population structure analysis by genotyping 40 unrelated SNPs. Distortions in the genotype frequencies between lung cancer sufferers and controls were identified using 2 by 3 contingency tables. Where the homozygote genotype (recessive model) or combined homozygote and heterozygote genotypes (codominant model) for the minor allele were found in excess in the healthy smokers controls compared to the lung cancer cohort, these SNP genotypes were assigned as protective. Where the homozygote genotype (recessive model) or combined homozygote and heterozygote genotypes (codominant model) for the minor allele were found in excess in the lung cancer cohort compared to healthy smokers controls, these SNP genotypes were assigned as susceptible. The magnitude of the effect from each SNP was analysed using univariate analysis and multivariate analysis. Based on these analyses, SNPs were ranked according to their ability to discriminate between lung cancer sufferers and controls, and combined as described to generate the SNP score. Non-genetic risk factors including age and family history were also analysed, and combined with the SNP score to generate a composite SNP score.

Results

Table 24 below summarises the univariate analysis showing protective and susceptibility SNPs associated with lung cancer as set out herein. Odd's ratios (OR) and p values are for cancer patients compared to resistant smokers with normal lung function. Table 24 also summarises the multivariate analysis, where stepwise regression analysis was performed and chi squared values are presented for each polymorphism.

TABLE 24
Genotypes and results of regression analysis—19 SNP panel

| | | Lung | Smoking | Call | Univariate | | Stepwise | | |
|---|---|---|---|---|---|---|---|---|---|
| SNP (rs#) | Genotype | cancer | controls | rate | OR | P value | regression χ2 | P value | Phenotype |
| CYP 2E1 | TT/TC | 24 (6%) | 14 (3%) | 95% | 2.1 | 0.03 | 0.63 | 0.24 | susceptibility |
| (Rsa 1 C/T) | CC | 379 (94%) | 463 (97%) | | (1.0-4.3) | | (0.29-1.37) | | |
| Interleukin-18 | CC | 237 (54%) | 208 (45%) | 96% | 1.4 | 0.009 | 0.65 | 0.007 | susceptibility |
| (−133 G/C) | CG/GG | 201 (46%) | 250 (55%) | | (1.1-1.9) | | (0.48-0.89) | | |
| Interleukin-8 | TT | 129 (31%) | 109 (23%) | 96% | 1.5 | 0.005 | 0.72 | 0.06 | susceptibility |
| (−251 A/T) | AT/AA | 284 (69%) | 367 (77%) | | (1.1-2.1) | | (0.51-1.02) | | |
| Interleukin 1B | GG | 215 (49%) | 212 (44%) | 99% | 1.2 | 0.14 | 0.86 | 0.33 | susceptibility |
| (rs 16944) | AA/AG | 224 (51%) | 269 (56%) | | (0.9-1.6) | | (0.63-1.17) | | |
| ITGA11 | AA | 14 (3%) | 6 (1%) | 98% | 2.6 | 0.04 | 0.28 | 0.02 | susceptibility |
| (rs2306022) | GA/GG | 422 (97%) | 470 (99%) | | (0.9-7.6) | | (0.10-0.84) | | |
| N-acetylcysteine | GG | 239 (56%) | 222 (47%) | 97% | 1.4 | 0.006 | 0.76 | 0.08 | susceptibility |
| transferase 2 | AA/AG | 189 (44%) | 253 (53%) | | (1.1-1.9) | | (0.56-1.03) | | |
| (rs 1799930) | | | | | | | | | |
| α1-antichymotrypsin | GG | 123 (28%) | 96 (20%) | 98% | 1.6 | 0.004 | 0.69 | 0.05 | susceptibility |
| (−15 A/G) | AG/AA | 312 (72%) | 383 (80%) | | (1.2-2.2) | | (0.48-0.99) | | |
| Cerberus 1 | AA/AG | 71 (16%) | 59 (12%) | 97% | 1.4 | 0.10 | 0.71 | 0.10 | susceptibility |
| (rs 10115703) | GG | 363 (84%) | 413 (88%) | | (0.9-2.0) | | 0.45-1.10 | | |
| DAT1 | GT/TT | 64 (15%) | 50 (10%) | 98% | 1.5 | 0.04 | 0.68 | 0.06 | susceptibility |
| (rs6413429) | GG | 367 (85%) | 431 (90%) | | (1.0-2.3) | | (0.43-1.10) | | |
| TNFR1 | AA | 148 (36%) | 142 (30%) | 96% | 1.3 | 0.05 | 0.88 | 0.20 | susceptibility |
| (rs1139417) | AG/GG | 258 (64%) | 329 (70%) | | (1.0-1.8) | | (0.64-1.23) | | |
| TLR9 | CC | 12 (3%) | 6 (1%) | 96% | 2.2 | 0.12 | 0.57 | 0.33 | susceptibility |
| (rs5743836) | CT/TT | 419 (97%) | 455 (99%) | | (0.8-6.6) | | (0.19-1.75) | | |
| P73 | CC | 219 (52%) | 292 (62%) | 96% | 0.65 | 0.001 | 1.50 | 0.01 | protective |
| (rs 2273953) | TC/TT | 206 (48%) | 178 (38%) | | (0.49-0.85) | | (1.1-2.04) | | |
| SOD3 | GG/GC | 4 (1%) | 15 (3%) | 96% | 0.28 | 0.02 | 8.43 | 0.01 | protective |
| (rs1799895) | CC | 425 (99%) | 451 (97%) | | (0.10-0.90) | | (1.65-43.22) | | |
| ITGB3 | GG/GA | 44 (10%) | 77 (16%) | 98% | 0.59 | 0.008 | 1.4 | 0.009 | protective |
| (rs2317676) | AA | 391 (90%) | 403 (84%) | | (0.39-0.89) | | (1.17-3.00) | | |
| DRD2 | CDel/Del.Del | 70 (16%) | 107 (22%) | 98% | 0.68 | 0.02 | 1.80 | 0.005 | protective |
| (rs 1799732) | CC | 359 (84%) | 372 (78%) | | (0.48-0.96) | | (1.20-2.70) | | |
| BCL2 | AA | 103 (24%) | 145 (31%) | 97% | 0.71 | 0.03 | 1.4 | 0.05 | protective |
| (rs 2279115) | AC/CC | 328 (76%) | 330 69%) | | (0.53-0.97) | | (1.01-2.04) | | |
| XPD | GG | 60 (14%) | 81 (18%) | 96% | 0.74 | 0.11 | 1.35 | 0.18 | protective |
| (rs 13181) | GT/TT | 376 (86%) | 377 (82%) | | (0.51-1.10) | | (0.90-2.10) | | |
| REV1 | CC | 128 (29%) | 163 (34%) | 98% | 0.79 | 0.10 | 1.34 | 0.08 | protective |
| (rs3087386) | TC/TT | 310 (71%) | 312 (66%) | | (0.59-1.10) | | (0.97-1.87) | | |
| FasL | TT | 53 (12%) | 78 (16%) | 98% | 0.72 | 0.09 | 1.46 | 0.10 | protective |
| (rs763110) | TC/CC | 379 (88%) | 403 (84%) | | (0.49-1.10) | | (0.93-2.29) | | |

Having defined the SNP panel SNP score, the genetic data was then analysed together with non-genetic data (specifically age, family history, history of COPD, and smoking exposure). Using multiple regression analysis, the magnitude of the effect of the 19 SNP panel in relation to age, family history and smoking exposure was determined A score for age (+4 for those over 60 years old), history of COPD (+4 for those with self reported COPD/emphysema) and family history (+3 to those with a first degree relative with lung cancer) was then assigned. As smoking exposure was a recruitment criteria, only a small contribution from smoking exposure was observed and was thus omitted from the composite SNP score. This SNP score was compared with (a) the frequency of lung cancer, and (b) the floating absolute relative risk among the combined smoking cohort.

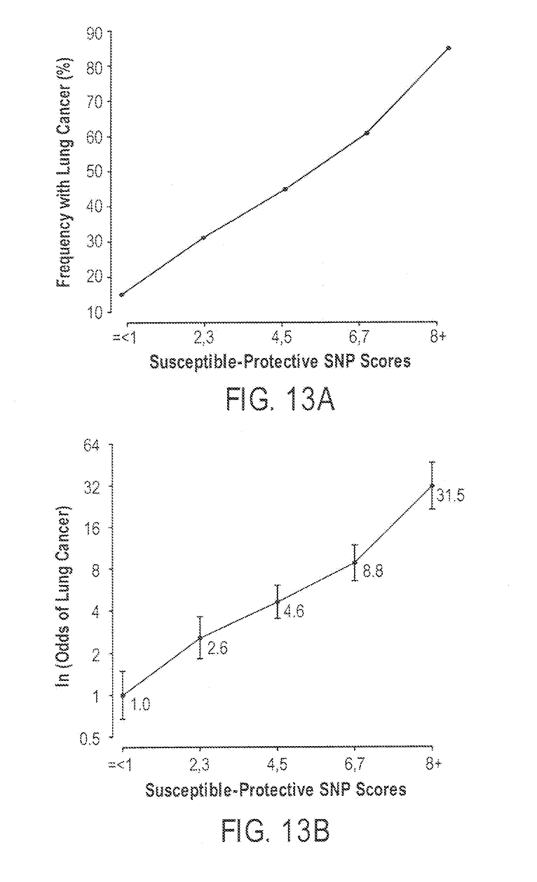

A linear relationship was observed across composite lung cancer SNP scores ≦1 to 8+ with lung cancer frequency spanning 15% to 85% (FIG. 13a). The magnitude of the effect was examined using the floating absolute risk plotted on a log scale (equivalent to an Odds ratio, OR), which references the lowest frequency group as 1 (referent group, lung cancer score ≦1) and compares each lung cancer score relative to the referent group (FIG. 13b). The OR ranged from 1 to 31.5 across the lung cancer scores when subjects are grouped roughly as quintiles. The OR was even higher for those with a SNP score of 9+.

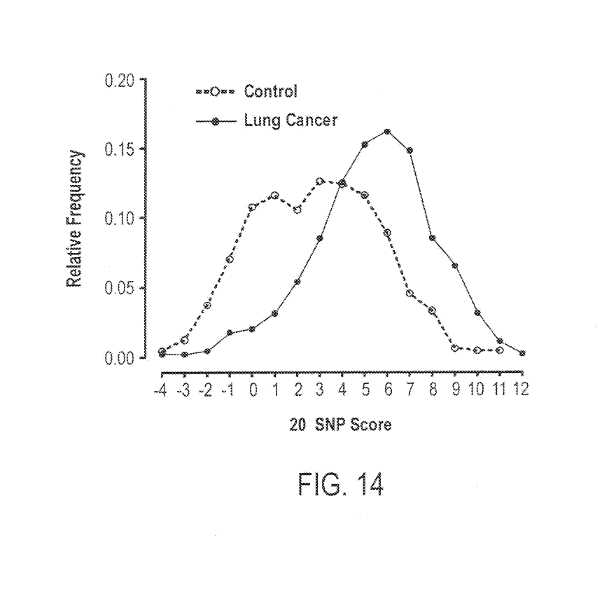

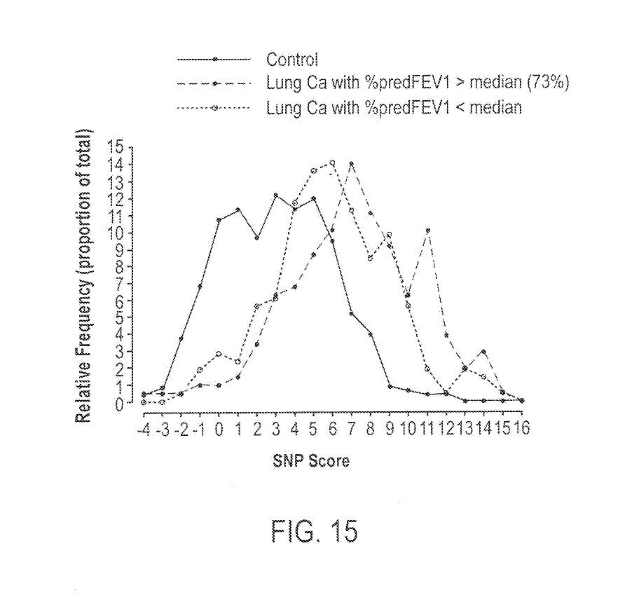

In a receiver operator curve analysis, the area under the curve (AUC, or C statistic) for the 19 SNP panel, age, family history of lung cancer, and history of COPD were 0.68, 0.70, 0.55, and 0.62, respectively. The distribution of the SNP score between cases and controls for the total cohort (n=930) shows a bimodal distribution (FIG. 14). Corresponding sensitivities and specificities on receiver-operator-curve analyses are shown in Table 25 below.

TABLE 25
Sensitivity and specificity estimates—19 SNP panel

| Lung cancer score | Sensitivity | 95% CI | Specificity | 95% CI |
|---|---|---|---|---|
| ≧1 | 95% | 94-98% | 23% | 19-27% |
| ≧3 | 89% | 86-92% | 44% | 39-48% |
| ≧7 | 50% | 45-55% | 89% | 86-91% |
| ≧9 | 28% | 23-32% | 98% | 96-99% |

Discussion

The composite SNP score derived from the 19 SNP panel in combination with non-genetic risk factores as described in this example generated a C statistic of 0.78, and a cut off of ≧3 with a sensitivity of 89% and corresponding specificity of 44%.

The C statistic for the SNP score derived from the 19 SNP panel in the absence of non-genetic risk factors was 0.70, indicating its useful predictive and discriminatory utility and suitability for use in the methods described herein, both on its own or in combination with non-genetic risk factors.

### Example 7

Table 26 below presents representative examples of polymorphisms in linkage disequilibrium with the polymorphisms specified herein. Examples of such polymorphisms can be located using public databases, such as that available at www.hapmap.org. Specified polymorphisms are shown in parentheses. The rs numbers provided are identifiers unique to each polymorphism.

TABLE 26
Polymorphism reported to be in LD with polymorphisms specified herein.
CAMKK1

| | | | | |
|---|---|---|---|---|
| rs11078470 | rs1029801 | rs11650638 | rs1029800 | (rs7214723) |
| rs6502751 | rs7214864 | rs9914305 | rs2058257 | rs8065798 |
| rs9904678 | rs7223713 | rs4790546 | rs7208983 | rs9898774 |
| rs7223709 | rs7212114 | rs11651131 | rs7221812 | rs12150410 |

| | |
|---|---|
| rs7221971 | rs9897177 |

ITGA11

| | | | | |
|---|---|---|---|---|
| rs11633421 | rs6494734 | rs898581 | rs1239019 | rs964691 |
| rs898580 | rs3736495 | rs8025985 | rs11072008 | rs3736494 |
| rs2306025 | rs12050550 | rs3736493 | rs2306024 | rs716379 |
| rs8041788 | rs2306023 | rs1380883 | rs8043152 | (rs2306022) |
| rs3784342 | rs16951774 | rs898586 | rs1380882 | rs1996361 |
| rs12442156 | rs3784344 | rs5016065 | rs7176011 | rs3784345 |
| rs2899735 | rs7176339 | rs11632266 | rs2414996 | rs898585 |
| rs1124577 | rs2414997 | rs4776395 | rs7177709 | rs7171871 |
| rs7182350 | rs3784346 | rs1516869 | rs12908869 | rs7180218 |
| rs16951777 | rs7161871 | rs748891 | rs16951778 | rs11632400 |
| rs748892 | rs3784335 | rs898584 | rs17266192 | rs17318470 |
| rs16951816 | rs898579 | rs3784336 | rs7179347 | rs12440936 |
| rs3784337 | rs7178537 | rs748971 | rs16951779 | rs7179545 |
| rs8029838 | rs898588 | rs2125998 | rs16951835 | rs7163918 |
| rs10162690 | rs8031003 | rs2271723 | rs9302249 | rs4776396 |
| rs898587 | rs7162991 | rs2306021 | rs1237911 | rs6494735 |
| rs16951841 | rs2271722 | rs4777040 | rs11072006 | rs6494736 |
| rs11630928 | rs8030178 | rs11635643 | rs8029230 | rs4777037 |
| rs8028967 | rs3736491 | rs8028971 | rs7176267 | rs8029113 |
| rs11072007 | rs4777041 | rs4777038 | rs8029452 | rs4777039 |
| rs7169899 | rs1533469 | rs4777042 | rs11858293 | rs8035990 |
| rs7179228 | rs2414998 | rs7179598 | rs16951819 | rs8042664 |
| rs2169214 | rs11852504 | rs12912832 | rs7167822 | rs2125997 |
| rs2292745 | rs7181259 | rs7168069 | rs1975874 | rs7169698 |
| rs898583 | rs6494733 | rs16951820 | rs970264 | rs898582 |

| | |
|---|---|
| rs1319223 | rs1563894 |

CER1

| | | | | |
|---|---|---|---|---|
| rs10810224 | rs17289263 | rs3761666 | rs13286013 | rs7022304 |
| rs7870750 | rs10961679 | rs7022400 | rs10121506 | rs10961680 |
| rs11999277 | rs10118242 | rs10961681 | rs1494360 | rs10118290 |
| rs951273 | rs1494359 | rs16932212 | rs2131883 | rs1494358 |
| rs11794846 | rs2131882 | rs1494357 | rs10122395 | rs12338263 |
| rs3747532 | rs10125285 | rs12338303 | (rs10115703) | rs1494351 |
| rs12338380 | rs10122490 | rs1494350 | rs2088042 | rs7018937 |
| rs10961683 | rs12347640 | rs12115314 | rs10961684 | rs10122817 |
| rs7035643 | rs11793334 | rs12115487 | rs10961682 | rs7019731 |
| rs11789968 | rs7019387 | rs10810225 | rs3761665 | rs3819004 |

| | | |
|---|---|---|
| rs10123442 | rs7036635 | rs10810226 |

XRCC4

| | | | | |
|---|---|---|---|---|
| rs36059813 | rs28360323 | rs10514256 | rs35770549 | rs28360322 |
| rs10514255 | rs35770061 | rs28360321 | rs10514254 | rs35704249 |
| rs28360320 | rs10434637 | rs35694031 | rs17567561 | rs10078343 |
| rs35618200 | rs17205881 | rs10070866 | rs35262280 | rs16900371 |
| rs10067830 | rs35219614 | rs16900367 | rs10061326 | rs35211331 |
| rs16900363 | rs10061086 | rs34801422 | rs16900362 | rs10057194 |
| rs34697956 | rs16900361 | rs10057054 | rs34646294 | rs16900359 |
| rs9293337 | rs34626079 | rs16900357 | rs9293336 | rs34544738 |
| rs16900353 | rs9293335 | rs34326210 | rs16900343 | rs7736592 |
| rs34164901 | rs16900342 | rs7735781 | rs34052855 | rs16900341 |
| rs7734849 | rs34006354 | rs16900340 | rs7729473 | rs28746479 |
| rs16900339 | rs7729020 | rs28746478 | rs16900330 | rs7728486 |
| rs28746477 | rs16900328 | rs7727606 | rs28746476 | rs16900325 |
| rs7716696 | rs28360351 | rs16900322 | rs7714809 | rs28360350 |
| rs16900317 | rs7711016 | rs28360349 | rs16900315 | rs6869679 |
| rs28360348 | rs13359237 | rs4987240 | rs28360347 | rs13358544 |
| rs4703951 | rs28360346 | rs13357939 | rs4703950 | rs28360345 |
| rs13187520 | rs4703568 | rs28360344 | rs13167490 | rs4438854 |
| rs28360343 | rs13167223 | rs3910950 | rs28360342 | rs13163691 |
| rs3836874 | rs28360341 | rs13163534 | rs3836873 | rs28360340 |
| rs13155538 | rs3777020 | rs28360339 | rs12697728 | rs3777019 |
| rs28360338 | rs12520831 | rs3777018 | rs28360337 | rs12186876 |
| rs3777015 | rs28360336 | rs11960030 | rs2891980 | rs28360335 |
| rs11960003 | rs2386275 | rs28360334 | rs11959198 | rs2084099 |
| rs28360333 | rs11958342 | rs2035990 | rs28360332 | rs11955413 |
| rs1805377 | rs28360331 | rs11954157 | (rs1056503) | rs28360330 |
| rs11953364 | rs382069 | rs28360329 | rs11950724 | rs301292 |
| rs28360328 | rs11749552 | rs301291 | rs28360327 | rs10805813 |
| rs177712 | rs28360326 | rs10805812 | rs28360325 | rs10642662 |

| | |
|---|---|
| rs28360324 | rs10514257 |

BRCA2

| | | | | | | |
|---|---|---|---|---|---|---|
| rs36116910 | rs28897730 | rs11571808 | rs11571701 | rs11571598 | rs7337784 | rs773032 |
| rs36114000 | rs28897729 | rs11571807 | rs11571700 | rs11571597 | rs7337574 | rs773031 |
| rs36091054 | rs28897728 | rs11571806 | rs11571699 | rs11571596 | rs7337016 | rs773030 |
| rs36073425 | rs28897727 | rs11571805 | rs11571698 | rs11571595 | rs7336403 | rs773029 |
| rs36060526 | rs28897726 | rs11571804 | rs11571697 | rs11571594 | rs7334543 | rs773027 |
| rs36018961 | rs28897725 | rs11571803 | rs11571696 | rs11571593 | rs7332492 | rs766173 |
| rs35979864 | rs28897724 | rs11571802 | rs11571695 | rs11571592 | rs7331638 | rs721185 |
| rs35930474 | rs28897723 | rs11571801 | rs11571694 | rs11571591 | rs7330025 | rs703224 |
| rs35768834 | rs28897722 | rs11571800 | rs11571693 | rs11571590 | rs7328654 | rs703223 |
| rs35697303 | rs28897721 | rs11571799 | rs11571692 | rs11571589 | rs7328264 | rs703213 |
| rs35685866 | rs28897720 | rs11571798 | rs11571691 | rs11571588 | rs7328101 | rs693963 |
| rs35628833 | rs28897719 | rs11571797 | rs11571690 | rs11571587 | rs7327867 | rs664345 |
| rs35596121 | rs28897718 | rs11571796 | rs11571689 | rs11571586 | rs7327813 | rs651906 |
| rs35573139 | rs28897717 | rs11571794 | rs11571688 | rs11571585 | rs7327677 | rs573014 |
| rs35571300 | rs28897716 | rs11571792 | rs11571687 | rs11571584 | rs7327471 | rs559067 |
| rs35563967 | rs28897715 | rs11571791 | rs11571686 | rs11571583 | rs7324145 | rs543304 |
| rs35527903 | rs28897714 | rs11571790 | rs11571685 | rs11571582 | rs7320990 | rs542551 |
| rs35497963 | rs28897713 | rs11571789 | rs11571684 | rs11571581 | rs7318434 | rs517118 |
| rs35486082 | rs28897712 | rs11571788 | rs11571683 | rs11571580 | rs6561306 | rs472817 |
| rs35477961 | rs28897711 | rs11571787 | rs11571682 | rs11571579 | rs5802644 | rs396579 |
| rs35408951 | rs28897710 | rs11571786 | rs11571681 | rs11571578 | rs4987117 | rs206346 |
| rs35382259 | rs28897709 | rs11571784 | rs11571680 | rs11571577 | rs4987049 | rs206344 |
| rs35335654 | rs28897708 | rs11571782 | rs11571679 | rs11571576 | rs4987048 | rs206343 |
| rs35324259 | rs28897707 | rs11571780 | rs11571678 | rs11571575 | rs4987047 | rs206342 |
| rs35315530 | rs28897706 | rs11571779 | rs11571676 | rs11571574 | rs4987046 | rs206341 |
| rs35188168 | rs28897705 | rs11571778 | rs11571675 | rs11552891 | rs4986860 | rs206340 |
| rs35069894 | rs28897704 | rs11571777 | rs11571674 | rs11464335 | rs4986859 | rs206319 |
| rs35029074 | rs28897703 | rs11571776 | rs11571673 | rs11460904 | rs4986858 | rs206318 |
| rs35027705 | rs28897702 | rs11571775 | rs11571672 | rs11451886 | rs4986856 | rs206147 |
| rs35005399 | rs28897701 | rs11571774 | rs11571671 | rs11426352 | rs4942505 | rs206146 |
| rs34959007 | rs28897700 | rs11571773 | rs11571670 | rs11371521 | rs4942499 | rs206145 |
| rs34943677 | rs28657708 | rs11571772 | rs11571669 | rs11327981 | rs4942486 | rs206123 |
| rs34926095 | rs28641896 | rs11571771 | rs11571668 | rs11312202 | rs4942485 | rs206122 |
| rs34925070 | rs28569916 | rs11571770 | rs11571667 | rs11306457 | rs4942448 | rs206121 |
| rs34895626 | rs28479757 | rs11571769 | rs11571666 | rs11291838 | rs4942443 | rs206120 |
| rs34891002 | rs28473213 | rs11571768 | rs11571665 | rs11147494 | rs4942440 | rs206099 |
| rs34842101 | rs17692629 | rs11571767 | rs11571664 | rs11147493 | rs4942439 | rs206098 |
| rs34841049 | rs17636116 | rs11571766 | rs11571663 | rs11147492 | rs4942423 | rs206097 |
| rs34835575 | rs17077554 | rs11571765 | rs11571662 | rs11147491 | rs4570704 | rs206096 |
| rs34816981 | rs17077542 | rs11571764 | rs11571661 | rs11147490 | rs3837580 | rs206095 |
| rs34809891 | rs17077541 | rs11571763 | rs11571660 | rs11147489 | rs3803282 | rs206081 |
| rs34770647 | rs17077519 | rs11571762 | rs11571659 | rs11147488 | rs3783265 | rs206080 |
| rs34704662 | rs13378910 | rs11571761 | rs11571658 | rs11147486 | rs3764792 | rs206079 |
| rs34692639 | rs13378905 | rs11571760 | rs11571657 | rs10870659 | rs3764791 | rs206078 |
| rs34647461 | rs13378423 | rs11571759 | rs11571656 | rs10577567 | rs3752451 | rs206077 |
| rs34578379 | rs13378422 | rs11571758 | rs11571655 | rs10492397 | rs3752448 | rs206076 |
| rs34578349 | rs12871316 | rs11571757 | rs11571654 | rs10492396 | rs3752447 | rs206075 |
| rs34575057 | rs12871310 | rs11571756 | rs11571653 | rs10492395 | rs3752446 | rs206074 |
| rs34469166 | rs12869544 | rs11571754 | rs11571652 | rs9943890 | rs3210648 | rs206073 |
| rs34437679 | rs12869093 | rs11571753 | rs11571651 | rs9943888 | rs3092990 | rs206072 |
| rs34380010 | rs12868315 | rs11571752 | rs11571650 | rs9943876 | rs3072043 | rs206071 |
| rs34370449 | rs12862392 | rs11571751 | rs11571649 | rs9634798 | rs3072042 | rs206070 |
| rs34355306 | rs12862064 | rs11571750 | rs11571648 | rs9634797 | rs3072040 | rs206069 |
| rs34351119 | rs12862049 | rs11571749 | rs11571647 | rs9634796 | rs2761367 | rs206068 |
| rs34345002 | rs12859126 | rs11571748 | rs11571646 | rs9634672 | rs2761363 | rs206067 |
| rs34309943 | rs12859094 | rs11571747 | rs11571644 | rs9595469 | rs2320236 | rs189979 |
| rs34288419 | rs12859079 | rs11571746 | rs11571643 | rs9595468 | rs2238163 | rs176176 |
| rs34273171 | rs12858763 | rs11571745 | rs11571642 | rs9595456 | rs2238162 | rs169548 |
| rs34225677 | rs12858735 | rs11571744 | rs11571641 | rs9595402 | rs2227944 | rs169547 |
| rs34184533 | rs12858723 | rs11571743 | rs11571640 | rs9595395 | rs2227943 | rs169546 |
| rs34178365 | rs12858361 | rs11571742 | rs11571639 | rs9590958 | rs2219594 | rs144848 |
| rs34175773 | rs12854843 | rs11571741 | rs11571638 | rs9590951 | rs2126042 | rs15869 |

| | | | | | |
|---|---|---|---|---|---|
| rs34108667 | rs12853807 | rs11571740 | rs11571637 | rs9590940 | rs2100785 |
| rs34102917 | rs12561064 | rs11571739 | rs11571636 | rs9590939 | rs1963505 |
| rs34080444 | rs12429216 | rs11571738 | rs11571635 | rs9590938 | rs1853521 |
| rs34075550 | rs12017223 | rs11571737 | rs11571634 | rs9567674 | rs1853520 |
| rs34009686 | rs11842816 | rs11571736 | rs11571633 | rs9567670 | rs1853519 |
| rs34001953 | rs11841349 | rs11571735 | rs11571632 | rs9567666 | rs1801499 |
| rs28897762 | rs11839855 | rs11571734 | rs11571631 | rs9567654 | rs1801439 |
| rs28897761 | rs11620336 | rs11571733 | rs11571630 | rs9567639 | rs1801426 |
| rs28897760 | rs11616673 | rs11571732 | rs11571629 | rs9567623 | rs1801406 |
| rs28897759 | rs11571837 | rs11571731 | rs11571628 | rs9567609 | rs1799968 |
| rs28897758 | rs11571836 | rs11571730 | rs11571627 | rs9567605 | rs1799956 |
| rs28897757 | rs11571835 | rs11571729 | rs11571626 | rs9567600 | rs1799955 |
| rs28897756 | rs11571834 | rs11571728 | rs11571625 | rs9567582 | rs1799954 |
| rs28897755 | (rs11571833) | rs11571727 | rs11571624 | rs9567578 | rs1799953 |
| rs28897754 | rs11571832 | rs11571726 | rs11571623 | rs9567576 | rs1799952 |
| rs28897753 | rs11571831 | rs11571725 | rs11571622 | rs9551726 | rs1799951 |
| rs28897752 | rs11571830 | rs11571723 | rs11571621 | rs9534367 | rs1799944 |
| rs28897751 | rs11571829 | rs11571722 | rs11571620 | rs9534344 | rs1475990 |
| rs28897750 | rs11571828 | rs11571721 | rs11571619 | rs9534342 | rs1460817 |
| rs28897749 | rs11571827 | rs11571720 | rs11571618 | rs9534323 | rs1460816 |
| rs28897748 | rs11571826 | rs11571719 | rs11571617 | rs9534318 | rs1380946 |
| rs28897747 | rs11571825 | rs11571718 | rs11571616 | rs9534286 | rs1207954 |
| rs28897746 | rs11571824 | rs11571717 | rs11571615 | rs9534275 | rs1207953 |
| rs28897745 | rs11571823 | rs11571716 | rs11571614 | rs9534274 | rs1207952 |
| rs28897744 | rs11571822 | rs11571715 | rs11571613 | rs9534270 | rs1148321 |
| rs28897743 | rs11571821 | rs11571714 | rs11571612 | rs9534269 | rs1148320 |
| rs28897742 | rs11571820 | rs11571713 | rs11571611 | rs9534268 | rs1128611 |
| rs28897741 | rs11571819 | rs11571712 | rs11571610 | rs9534262 | rs1128610 |
| rs28897740 | rs11571818 | rs11571711 | rs11571609 | rs9534259 | rs1062947 |
| rs28897739 | rs11571817 | rs11571710 | rs11571608 | rs9534174 | rs1062946 |
| rs28897738 | rs11571816 | rs11571709 | rs11571607 | rs9526165 | rs1046984 |
| rs28897737 | rs11571815 | rs11571708 | rs11571606 | rs9526160 | rs1045789 |
| rs28897736 | rs11571814 | rs11571707 | rs11571605 | rs9526148 | rs1029304 |
| rs28897735 | rs11571813 | rs11571706 | rs11571604 | rs9526131 | rs1012130 |
| rs28897734 | rs11571812 | rs11571705 | rs11571603 | rs7992196 | rs1012129 |
| rs28897733 | rs11571811 | rs11571704 | rs11571602 | rs7982943 | rs811637 |
| rs28897732 | rs11571810 | rs11571703 | rs11571601 | rs7981512 | rs798652 |
| rs28897731 | rs11571809 | rs11571702 | rs11571600 | rs7491644 | rs773033 |

P73

| | | | | | |
|---|---|---|---|---|---|
| rs3765702 | rs1122638 | rs3819955 | rs5031051 | rs3753205 | rs3765703 |
| rs12062249 | rs3765707 | rs5031052 | rs3765714 | rs10910007 | rs2368542 |
| rs12059298 | (rs2273953) | rs3765715 | rs12028205 | rs6665164 | rs3765708 |
| rs1801173 | rs3765716 | rs12057230 | rs7554226 | rs12025725 | rs4648547 |
| rs1122723 | rs12024891 | rs10910009 | rs3765709 | rs1122724 | rs10910008 |
| rs1885874 | rs3765710 | rs1122725 | rs12121199 | rs12403618 | rs3765711 |
| rs12095743 | rs3765705 | rs12403927 | rs3765712 | rs1122639 | rs3765706 |

| | |
|---|---|
| rs10910010 | rs3765713 |

CYP3A43

| | | | | | |
|---|---|---|---|---|---|
| rs2738258 | rs1041966 | rs2023548 | rs688926 | rs13236744 | rs2687110 |
| rs12721632 | rs493380 | rs687134 | rs13444455 | rs17294659 | rs12721636 |
| rs1554511 | rs4236544 | rs10241225 | rs12670850 | rs12721633 | rs667660 |
| rs4646472 | rs10225908 | rs6970689 | rs12721637 | rs620020 | rs671673 |
| rs2897018 | rs11768200 | rs1800713 | rs17161937 | rs660629 | rs528144 |
| rs6465753 | rs2740574 | rs1403195 | rs533486 | rs6975773 | rs4986914 |
| rs473706 | rs501275 | rs2263430 | rs2740573 | rs10255255 | rs641815 |
| rs6415332 | rs11773597 | rs585071 | rs641761 | rs2263431 | rs1851426 |
| rs2023165 | rs472667 | rs2687106 | rs12114000 | rs1036374 | rs579424 |
| rs10270146 | rs2740572 | rs651430 | rs13234698 | rs12721619 | rs4301384 |
| rs653245 | rs549061 | rs12721625 | rs2740571 | rs7807561 | rs545400 |
| rs3800957 | rs2687103 | rs800675 | rs4646474 | rs7801671 | rs7811022 |
| rs558112 | rs487813 | rs16867648 | rs7811025 | rs558002 | rs1077078 |
| rs2687105 | rs2740570 | (C74 delA) | rs679320 | rs2687104 | rs4729550 |
| rs13236405 | rs678040 | rs10264769 | rs3958412 | rs800674 | rs568859 |
| rs2405184 | rs1320390 | rs800673 | rs800667 | rs2740575 | rs1320389 |
| rs523407 | rs6960775 | rs2253498 | rs2687102 | rs642761 | rs565079 |
| rs2253493 | rs2687101 | rs496000 | rs675644 | rs17161904 | rs2740569 |
| rs800672 | rs648515 | rs4602816 | rs2687100 | rs4268042 | rs800666 |
| rs3991692 | rs2737418 | rs892753 | rs646563 | rs6957392 | rs760368 |
| rs12671336 | rs694939 | rs12721634 | rs2017121 | rs2164226 | rs800664 |

BCL2

| | | | | | |
|---|---|---|---|---|---|
| rs12458289 | rs1473418 | rs2551407 | rs2849372 | rs949037 | (rs2279115) |
| rs10460159 | rs2615196 | rs2849380 | rs2551400 | rs2849383 | rs2849371 |
| rs1462128 | rs2551401 | rs11663788 | rs8098151 | rs1462129 | rs7243985 |
| rs2849367 | rs3786327 | rs2051424 | rs2551402 | rs6810 | rs2850757 |
| rs2051423 | rs8099294 | rs2615201 | rs2850756 | rs1944422 | rs2051422 |
| rs736223 | rs2551410 | rs2085958 | rs1944423 | rs898891 | rs1893805 |
| rs12455492 | rs11659773 | rs2850767 | rs2032343 | rs7239542 | rs2551403 |
| rs2850768 | rs11152379 | rs1541295 | rs2551404 | rs2551408 | rs1541296 |
| rs4987712 | rs11660715 | rs2236719 | rs1809319 | rs1893806 | rs17687494 |
| rs2849376 | rs2003149 | rs4987711 | rs8094041 | rs2849375 | rs439670 |
| rs4987710 | rs2551405 | rs12327344 | rs489520 | rs1800477 | rs2850764 |
| rs2255302 | rs3744939 | rs1801018 | rs10460158 | rs12953721 | rs428356 |
| rs4987707 | rs2551406 | rs8083276 | rs383770 | rs4987706 | rs698708 |

rs7231949
ITGB3

| | | | | | |
|---|---|---|---|---|---|
| rs884696 | rs8074348 | rs951351 | rs13380810 | rs9303533 | rs7219925 |
| rs16941796 | rs7218632 | rs7223956 | rs7214993 | rs10514919 | rs2015729 |
| rs11651736 | rs7220606 | rs8075031 | rs11870334 | rs16941801 | rs3785870 |
| rs12162128 | rs1051452 | rs11870365 | rs7217214 | rs16941829 | rs7224753 |
| rs16941864 | rs16941776 | rs16941802 | rs2292864 | rs7221196 | (rs2317676) |
| rs16941780 | rs7212751 | rs12940355 | rs12603582 | rs3809865 | rs11657517 |
| rs11649785 | rs12951133 | rs12603725 | rs9916007 | rs11658221 | rs1000232 |
| rs12942670 | rs10221263 | rs8068200 | rs8073827 | rs2292866 | rs12943780 |
| rs12602240 | rs9894860 | rs12941431 | rs2292867 | rs12942968 | rs11870252 |
| rs12600603 | rs11651758 | rs16941807 | rs12951679 | rs11867253 | rs9893410 |
| rs12453200 | rs11079770 | rs12942997 | rs11867192 | rs7209109 | rs11651904 |
| rs8073229 | rs12943005 | rs3785873 | rs7225700 | rs11656865 | rs11868912 |
| rs13306482 | rs9747605 | rs4968313 | rs7503748 | rs1878067 | rs1969268 |
| rs9906248 | rs2317677 | rs11657963 | rs988684 | rs1533409 | rs3760372 |
| rs11658426 | rs984370 | rs5918 | rs15908 | rs13306488 | rs11650072 |
| rs8080254 | rs5920 | rs12709459 | rs13306489 | rs11079772 | rs8074094 |
| rs13306485 | rs13306483 | rs1969267 | rs12600865 | rs8066295 | rs12709458 |
| rs2292863 | rs8081202 | rs4968314 | rs11870620 | rs13306486 | rs5921 |
| rs16941855 | rs7218813 | rs11079769 | rs5917 | rs4642 | rs9914944 |
| rs9899121 | rs4486970 | rs2292699 | rs13306487 | rs11869835 | rs10853089 |
| rs3851806 | rs2292700 | rs4634 | rs12950632 | rs6504833 | rs16941793 |
| rs8064853 | rs7214096 | rs3744452 | rs7209700 | rs9912177 | rs7217710 |
| rs3744453 | rs4968312 | rs13306484 | rs7214468 | rs11868344 | rs8078614 |
| rs12451759 | rs11656809 | rs11870781 | rs3851807 | rs5919 | rs999323 |
| rs16941861 | rs12940207 | rs13306476 | rs3785872 | rs3809863 | rs8064871 |
| rs13306477 | rs12949936 | rs11655943 | rs8069732 | rs13306478 | rs11079771 |
| rs16941863 | rs11868894 | rs2292865 | rs11650022 | rs9674670 | rs8077753 |

| | | |
|---|---|---|
| rs13306480 | rs7211018 | rs9284377 |

DAT1

| | | | | | |
|---|---|---|---|---|---|
| rs2937639 | rs2447848 | rs11564751 | rs2617592 | rs1354139 | rs2550961 |
| rs2447847 | rs4029364 | rs2617591 | rs2652505 | rs2550962 | rs2516289 |
| rs4029363 | rs2652508 | rs11747778 | rs11564757 | rs2617601 | rs2937637 |
| rs2617590 | rs2078247 | rs2550963 | rs2735855 | rs2937636 | rs2617589 |
| rs2617584 | rs2937638 | rs3776485 | rs7733388 | rs2471921 | rs2550939 |
| rs1316830 | rs2550967 | rs11564750 | rs2617588 | rs2113330 | rs2735859 |
| rs2617600 | rs2550956 | rs2550949 | rs2975224 | rs2735858 | rs2735854 |
| rs11564749 | rs2652506 | rs2617583 | rs2859604 | rs2735935 | rs2652510 |
| rs10070282 | rs12652860 | rs2550965 | rs2735934 | rs2937635 | rs10079467 |
| rs12654851 | rs2516291 | rs2617599 | rs2975225 | rs2550947 | rs6879432 |
| rs2447850 | rs2975227 | rs3756450 | rs2550946 | rs9312868 | rs2516290 |
| rs2975226 | rs2617595 | rs2550945 | rs1478435 | rs2550966 | rs2652513 |
| rs2652509 | rs2550944 | rs1478434 | rs2254255 | rs2652512 | rs2617594 |
| rs250694 | rs10063727 | rs2963238 | rs456323 | rs2550955 | rs2550943 |
| rs4639276 | rs2617603 | rs2617598 | rs2550954 | rs565988 | rs2911493 |
| rs2735853 | rs2550953 | rs565985 | rs2471926 | rs2735852 | rs2550952 |
| rs250693 | rs2447849 | rs2617597 | rs2550951 | rs2550941 | rs2617602 |
| rs2652511 | rs2550950 | rs250692 | rs11564752 | rs2617596 | rs2963236 |
| rs193941 | rs2735857 | rs2550957 | rs2617593 | rs565123 | rs2735856 |

| | | |
|---|---|---|
| (rs6413429) | rs193942 | rs2550940 |

TNFR1

| | | | | | |
|---|---|---|---|---|---|
| rs1800693 | rs4149636 | rs4149581 | rs4149625 | rs4149618 | rs4149642 |
| rs2363888 | rs4149580 | rs4149571 | rs4149617 | rs4149641 | rs4149635 |
| rs4149579 | rs4149624 | rs12300705 | rs4149587 | rs877249 | rs4149578 |
| rs4149623 | rs11064143 | rs4149640 | rs4149583 | rs4149577 | rs4149622 |
| rs7297961 | rs12832171 | rs4149634 | rs4149627 | rs4441073 | rs11064145 |
| rs11525582 | rs2284344 | rs4149626 | rs767455 | rs11608320 | rs4149586 |
| rs4149633 | rs10774425 | (rs1139417) | rs11608322 | rs4149639 | rs4149632 |
| rs11836766 | rs2234649 | rs2228576 | rs12317730 | rs4149631 | rs4149576 |
| rs4149621 | rs1800692 | rs4149630 | rs4149575 | rs4149570 | rs4149638 |
| rs887477 | rs4149574 | rs16932532 | rs4149585 | rs4149629 | rs4149573 |
| rs4149620 | rs4149584 | rs4149582 | rs4149572 | rs4149619 | rs4149637 |

| | | |
|---|---|---|
| rs1860545 | rs11615387 | rs4149569 |

DRD2

| | | | | | |
|---|---|---|---|---|---|
| rs17529477 | rs4337071 | rs5013062 | rs12099213 | rs12361261 | rs17601612 |
| rs4630328 | rs12364283 | rs7934294 | rs4466875 | rs11214610 | rs11214612 |
| rs11214617 | rs12574578 | rs7131411 | rs4245146 | rs11214613 | rs7110440 |
| rs11301285 | rs4429089 | rs4245147 | rs11214614 | rs12808668 | rs17602285 |
| rs4245153 | rs4936270 | rs4350392 | rs12785817 | rs11214627 | rs4245154 |
| rs4936271 | rs11601054 | rs4483623 | rs10891564 | rs11214636 | rs4936272 |
| rs7930567 | rs10891556 | rs6589379 | rs4938026 | rs4274224 | rs12225915 |
| rs4424703 | rs4938023 | rs2002229 | rs4245148 | rs10891553 | rs11214618 |
| rs4503578 | rs2002228 | rs4245149 | rs12421616 | rs12800185 | rs4254099 |
| rs12280961 | rs12576411 | rs6589377 | rs7121986 | rs7111031 | rs12291458 |
| rs7109897 | rs7102650 | rs6589381 | rs4938024 | rs2514218 | rs17115596 |
| rs7939472 | rs11214619 | rs11214642 | rs2511514 | rs12805897 | rs11214615 |
| rs4482060 | rs6589382 | rs12417718 | rs4581480 | rs4938019 | rs10891562 |
| rs4245151 | rs7122454 | rs11214633 | rs4421776 | rs7949802 | rs7948028 |
| rs11214616 | rs11214623 | rs4533070 | rs10891550 | rs10891554 | rs4611239 |
| rs11214634 | rs7131056 | rs4938025 | rs4245150 | rs12275979 | rs11214611 |
| rs10789943 | rs17602038 | rs10789944 | rs4936274 | rs3935565 | rs4938021 |
| rs7928940 | rs12291794 | rs12418281 | rs4936275 | rs4479021 | rs4648317 |
| rs7116768 | rs4936276 | (rs1799732) | rs7109615 | rs12281924 | rs1986665 |
| rs7479729 | rs10891551 | rs4447205 | rs12363546 | rs7106947 | rs4322431 |
| rs1799978 | rs12576181 | rs5013060 | rs7117915 | rs5013059 | rs10736466 |
| rs1984739 | rs10891552 | rs4534613 | rs4938022 | rs4245152 | rs7118174 |

| | |
|---|---|
| rs5013061 | rs12292637 |

FasL

| | | | | | |
|---|---|---|---|---|---|
| rs1894626 | rs2859235 | rs2639617 | rs3021335 | rs16844867 | rs2639622 |
| rs10912122 | rs2859239 | rs2933547 | rs9787393 | rs2639621 | rs2639618 |
| rs2639616 | rs2859244 | rs9787248 | rs2859228 | rs2859236 | rs2131373 |
| rs2859245 | rs12080307 | rs2859229 | rs10798130 | rs12130118 | rs10753023 |
| rs749154 | rs1492899 | rs16844856 | rs2859240 | rs10798133 | rs749155 |
| rs12082528 | rs2021839 | rs2639615 | rs2859246 | (rs763110) | rs4304626 |
| rs2021838 | rs2859241 | rs2859247 | rs2859233 | rs2859237 | rs2859242 |

| | | | | |
|---|---|---|---|---|
| rs2639614 | rs2859234 | rs2859238 | rs2859243 | rs2859248 |

TLR9

| | | | | | |
|---|---|---|---|---|---|
| rs353551 | rs352168 | rs17052020 | rs5743847 | rs5743838 | rs352158 |
| rs352167 | rs10212560 | rs445676 | rs5743837 | rs614288 | rs352166 |
| rs12629425 | rs5743846 | (rs5743836) | rs6767333 | rs13064414 | rs9816466 |
| rs5743845 | rs187084 | rs9828488 | rs352165 | rs7614535 | rs352140 |
| rs352173 | rs11712164 | rs352162 | rs5743844 | rs352172 | rs17052017 |
| rs5743850 | rs5743843 | rs3774412 | rs9813448 | rs6809796 | rs5743842 |
| rs709315 | rs9813468 | rs13080616 | rs352139 | rs352171 | rs352164 |
| rs13060808 | rs5743841 | rs352170 | rs352163 | rs5743849 | rs5743840 |

| | | | |
|---|---|---|---|
| rs352169 | rs164640 | rs5743848 | rs5743839 |

### INDUSTRIAL APPLICATION

The present invention is directed to methods for assessing a subject's risk of developing lung cancer. The methods comprise the analysis of polymorphisms herein shown to be associated with increased or decreased risk of developing lung cancer, or the analysis of results obtained from such an analysis. The use of polymorphisms herein shown to be associated with increased or decreased risk of developing lung cancer in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

### PUBLICATIONS

Alberg A J, Samet J M. Epidemiology of lung cancer. Chest 2003, 123, 21s-49s.
Anthonisen N R. Prognosis in COPD: results from multi-center clinical trials. Am Rev Respir Dis 1989, 140, s95-s99.
Kuller L H, et al. Relation of forced expiratory volume in one second to lung cancer mortality in the MRFIT. Am J Epidmiol 1190, 132, 265-274.
Mayne S T, et al. Previous lung disease and risk of lung cancer among men and women nonsmokers. Am J Epidemiol 1999, 149, 13-20.
Nomura a, et al. Prospective study of pulmonary function and lung cancer. Am Rev Respir Dis 1991, 144, 307-311.
Schwartz A G. Genetic predisposition to lung cancer. Chest 2004, 125, 86s-89s.
Skillrud D M, et al. Higher risk of lung cancer in COPD: a prospective matched controlled study. Ann Int Med 1986, 105, 503-507.
Tockman M S, et al. Airways obstruction and the risk for lung cancer. Ann Int Med 1987, 106, 512-518.
Wu X, Zhao H, Suk R, Christiani D C. Genetic susceptibility to tobacco-related cancer. Oncogene 2004, 23, 6500-6523.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of” and “consisting of” may be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of determining a subject's risk of developing lung cancer comprising
analysing a sample from said subject for the presence or absence of one or more polymorphisms selected from the group consisting of:
Ser307Ser G/T (rs1056503) in the X-ray repair complementing defective repair in Chinese hamster cells 4 gene,
A/T c74delA in the gene encoding cytochrome P450 polypeptide CYP3A43;
A/C (rs2279115) in the gene encoding B-cell CLL/lymphoma 2;
A/G at +3100 in the 3′UTR (rs2317676) of the gene encoding Integrin beta 3;
−3714 G/T (rs6413429) in the gene encoding Dopamine transporter 1;
A/G (rs1139417) in the gene encoding Tumor necrosis factor receptor 1;
C/Del (rs1799732) in the gene encoding Dopamine receptor D2;
C/T (rs763110) in the gene encoding Fas ligand;
C/T (rs5743836) in the gene encoding Toll-like receptor 9; or
one or more polymorphisms in linkage disequilibrium with one or more of said polymorphisms, wherein the presence or absence of said polymorphism is indicative of the subject's risk of developing lung cancer.

2.-68. (canceled)


SEQUENCE LISTING (72 sequences)

SEQ ID NO: 1 - DNA, 30 bp, Artificial
  Synthetic
acgttggatgctgaattctcctcagatgac

SEQ ID NO: 2 - DNA, 30 bp, Artificial
  Synthetic
acgttggatgaatgcaagttcttcgtcagc

SEQ ID NO: 3 - DNA, 30 bp, Artificial
  Synthetic
acgttggatgaaaactcagacaccaggagc

SEQ ID NO: 4 - DNA, 30 bp, Artificial
  Synthetic
acgttggatgagatcaagaatgagcccgtg

SEQ ID NO: 5 - DNA, 30 bp, Artificial
  Synthetic
acgttggatgcctcttatttcagctgctgg

SEQ ID NO: 6 - DNA, 30 bp, Artificial
  Synthetic
acgttggatgagagaactctgattctggcg

SEQ ID NO: 7 - DNA, 30 bp, Artificial
  Synthetic
acgttggatgaccttgcccgtgtggttgaa

SEQ ID NO: 8 - DNA, 30 bp, Artificial
  Synthetic
acgttggatgtggcagggtacacagtcaca

SEQ ID NO: 9 - DNA, 30 bp, Artificial
  Synthetic
acgttggatgctgctgtttctcagagtttc

SEQ ID NO: 10 - DNA, 30 bp, Artificial
  Synthetic
acgttggatggcctgattcttcactacctg

SEQ ID NO: 11 - DNA, 18 bp, Artificial
  Synthetic
cctcagatgactccattt

SEQ ID NO: 12 - DNA, 19 bp, Artificial
  Synthetic
cctcagatgactccattta

SEQ ID NO: 13 - DNA, 24 bp, Artificial
  Synthetic
tgttcccctgggtggacaactcac

SEQ ID NO: 14 - DNA, 25 bp, Artificial
  Synthetic
tgttcccctgggtggacaactcacc

SEQ ID NO: 15 - DNA, 26 bp, Artificial
  Synthetic
tactcctgcctctaggaaagaccaca

SEQ ID NO: 16 - DNA, 27 bp, Artificial
  Synthetic
tactcctgcctctaggaaagaccacac

SEQ ID NO: 17 - DNA, 16 bp, Artificial
  Synthetic
ccctgcctggaggaca

SEQ ID NO: 18 - DNA, 17 bp, Artificial
  Synthetic
ccctgcctggaggacac

SEQ ID NO: 19 - DNA, 19 bp, Artificial
  Synthetic
ctgagatgtgctccttttt

SEQ ID NO: 20 - DNA, 20 bp, Artificial
  Synthetic
ctgagatgtgctcctttttc

SEQ ID NO: 21 - DNA, 19 bp, Artificial
  Synthetic
cctcagatgactccatttt

SEQ ID NO: 22 - DNA, 25 bp, Artificial
  Synthetic
tgttcccctgggtggacaactcact

SEQ ID NO: 23 - DNA, 27 bp, Artificial
  Synthetic
tactcctgcctctaggaaagaccacat

SEQ ID NO: 24 - DNA, 17 bp, Artificial
  Synthetic
ccctgcctggaggacat

SEQ ID NO: 25 - DNA, 20 bp, Artificial
  Synthetic
ctgagatgtgctccttttta

SEQ ID NO: 26 - DNA, 30 bp, Artificial
  Synthetic
acgttggatgtgctcaggtgtcattccttc

SEQ ID NO: 27 - DNA, 29 bp, Artificial
  Synthetic
acgttggatgggtggactgggccatcttc

SEQ ID NO: 28 - DNA, 30 bp, Artificial
  Synthetic
acgttggatgttctgtaacctggctttctc

SEQ ID NO: 29 - DNA, 30 bp, Artificial
  Synthetic
acgttggatgccaggaattcccagcttctt

SEQ ID NO: 30 - DNA, 30 bp, Artificial
  Synthetic
acgttggatgcaaaacaagggatggcggaa

SEQ ID NO: 31 - DNA, 30 bp, Artificial
  Synthetic
acgttggatgaaaggagctgtacctcctcg

SEQ ID NO: 32 - DNA, 30 bp, Artificial
  Synthetic
acgttggatgatcagaagaggattcctgcc

SEQ ID NO: 33 - DNA, 29 bp, Artificial
  Synthetic
acgttggatgttcacgcctccccaggaga

SEQ ID NO: 34 - DNA, 30 bp, Artificial
  Synthetic
acgttggatgtatgaactgggagatgctgg

SEQ ID NO: 35 - DNA, 30 bp, Artificial
  Synthetic
acgttggatgtgttgggagtgaggatgtct

SEQ ID NO: 36 - DNA, 30 bp, Artificial
  Synthetic
acgttggatgttgggatgtgctgttccctc

SEQ ID NO: 37 - DNA, 30 bp, Artificial
  Synthetic
acgttggatgagcagagacataatggaggc

SEQ ID NO: 38 - DNA, 30 bp, Artificial
  Synthetic
acgttggatgtgtcaggaggccttcaggtg

SEQ ID NO: 39 - DNA, 30 bp, Artificial
  Synthetic
acgttggatggttttatgagggcactggtc

SEQ ID NO: 40 - DNA, 30 bp, Artificial
  Synthetic
acgttggatgaggccatagctgtctggcat

SEQ ID NO: 41 - DNA, 29 bp, Artificial
  Synthetic
acgttggatgttccctttgtccctggtct

SEQ ID NO: 42 - DNA, 30 bp, Artificial
  Synthetic
acgttggatgaggctgcaaaccagtggaac

SEQ ID NO: 43 - DNA, 30 bp, Artificial
  Synthetic
acgttggatgctgggcaaacaatgaaaatg

SEQ ID NO: 44 - DNA, 17 bp, Artificial
  Synthetic
cttccttcctgcagagg

SEQ ID NO: 45 - DNA, 18 bp, Artificial
  Synthetic
cttccttcctgcagagga

SEQ ID NO: 46 - DNA, 24 bp, Artificial
  Synthetic
ggctttctcttttattttatagtt

SEQ ID NO: 47 - DNA, 25 bp, Artificial
  Synthetic
ggctttctcttttattttatagttc

SEQ ID NO: 48 - DNA, 21 bp, Artificial
  Synthetic
cccaacccctcctacccgttc

SEQ ID NO: 49 - DNA, 22 bp, Artificial
  Synthetic
cccaacccctcctacccgttcc

SEQ ID NO: 50 - DNA, 17 bp, Artificial
  Synthetic
ggctccttcatcgtccc

SEQ ID NO: 51 - DNA, 18 bp, Artificial
  Synthetic
ggctccttcatcgtcccc

SEQ ID NO: 52 - DNA, 24 bp, Artificial
  Synthetic
gatgctggtacatcccccaggcca

SEQ ID NO: 53 - DNA, 25 bp, Artificial
  Synthetic
gatgctggtacatcccccaggccac

SEQ ID NO: 54 - DNA, 17 bp, Artificial
  Synthetic
gctgttccctctgcctg

SEQ ID NO: 55 - DNA, 18 bp, Artificial
  Synthetic
gctgttccctctgcctga

SEQ ID NO: 56 - DNA, 17 bp, Artificial
  Synthetic
ggagggctccaccctga

SEQ ID NO: 57 - DNA, 18 bp, Artificial
  Synthetic
ggagggctccaccctgag

SEQ ID NO: 58 - DNA, 17 bp, Artificial
  Synthetic
cctgacctgctgctgcc

SEQ ID NO: 59 - DNA, 18 bp, Artificial
  Synthetic
cctgacctgctgctgcca

SEQ ID NO: 60 - DNA, 25 bp, Artificial
  Synthetic
aacccacagagctgctttgtatttc

SEQ ID NO: 61 - DNA, 26 bp, Artificial
  Synthetic
aacccacagagctgctttgtatttca

SEQ ID NO: 62 - DNA, 18 bp, Artificial
  Synthetic
cttccttcctgcagaggg

SEQ ID NO: 63 - DNA, 25 bp, Artificial
  Synthetic
ggctttctcttttattttatagtta

SEQ ID NO: 64 - DNA, 22 bp, Artificial
  Synthetic
cccaacccctcctacccgttca

SEQ ID NO: 65 - DNA, 18 bp, Artificial
  Synthetic
ggctccttcatcgtccca

SEQ ID NO: 66 - DNA, 25 bp, Artificial
  Synthetic
gatgctggtacatcccccaggccat

SEQ ID NO: 67 - DNA, 18 bp, Artificial
  Synthetic
gctgttccctctgcctgg

SEQ ID NO: 68 - DNA, 18 bp, Artificial
  Synthetic
ggagggctccaccctgat

SEQ ID NO: 69 - DNA, 18 bp, Artificial
  Synthetic
cctgacctgctgctgccg

SEQ ID NO: 70 - DNA, 26 bp, Artificial
  Synthetic
aacccacagagctgctttgtatttcg

SEQ ID NO: 71 - DNA, 25 bp, Artificial
  Synthetic
ggctttctcttttattttatagttg

SEQ ID NO: 72 - DNA, 25 bp, Artificial
  Synthetic
ggctttctcttttattttatagttt